(19) United States
(12) Patent Application Publication
(10) Pub. No.: US 2022/0230704 A1
(43) Pub. Date: Jul. 21, 2022

(54) DNA METHYLATION BASED HIGH RESOLUTION CHARACTERIZATION OF MICROBIOME USING NANOPORE SEQUENCING

(71) Applicant: Icahn School of Medicine at Mount Sinai, New York, NY (US)

(72) Inventors: Gang Fang, New York, NY (US); Alan Tourancheau, New York, NY (US)

(21) Appl. No.: 17/617,070

(22) PCT Filed: Jun. 12, 2020
(86) PCT No.: PCT/US20/37507

§ 371 Date: Dec. 7, 2021

Related U.S. Application Data
(60) Provisional application No. 62/860,952, filed on Jun. 13, 2019.

(51) Int. Cl. G16B 20/20

(52) U.S. Cl. CPC G16B 20/20

(57) ABSTRACT

Disclosed herein are computer-implemented methods of deconvoluting metagenomic assembled contigs from a microbiome sample and methods of using such for therapeutic, diagnostic, and environmental purposes.

### CROSS REFERENCE TO RELATED APPLICATIONS

The present application is a National Stage Application of International Application No. PCT/US2020/037507, filed Jun. 12, 2020 and published as WO 2020/252320 on Dec. 17, 2020, which claims the benefit of U.S. Provisional Application 62/860,952 filed Jun. 13, 2019, the disclosures of which are hereby incorporated by reference in their entirety.

### SEQUENCE LISTING

The ASCII file, entitled 093698-707271_P-181112-US_Sequence_Listing_ST25.txt, comprising 23.9 kilobytes (KB), submitted herewith is incorporated herein by reference. The corrected Sequence Listing contains the same sequence information forming part of the original application.

### FIELD

The present disclosure generally relates to computer-implemented methods for deconvoluting metagenomic assembled contigs from a microbiome sample using nanopore sequencing.

### BACKGROUND

Microbiomes, communities of bacteria, viruses, and other microbes, can be found in and on all known multicellular organisms. The ability to characterize microbiome communities may have important implications for understanding and manipulating ecosystem processes such as nutrient cycling, organic matter turnover, and the development or inhibition of soil pathogens. Further, opportunities for managing ecosystem services and bioprospecting soil microbial metabolism can be possible with a greater comprehension of how soil microbiomes interact under different conditions. Characterization of environmental microbiomes can aid in the understanding of a variety of ecological concerns ranging from the impact of soil microbes on the productivity of natural plant communities and agroecosystems to predicting waterborne disease risk in vulnerable water and sanitation infrastructures.

Recent studies have also implicated that disruption of a natural microbiome can result in serious health conditions including infectious diseases, cancers, and complex disorders such as Crohn's disease, ulcerative colitis, cancers, and diabetes, among many others. Interventional manipulations of the microbiota, either by probiotics, prebiotics and/or fecal transplantation, are realistic therapeutic strategies for such conditions after the microbiome community has been characterized. Additionally, emerging evidence indicates that the gut microbiome is a vital and sensitive indicator for predicting abnormal health status of humans, suggesting that characterization of a subject's microbiome can be used in early disease diagnosis and prevention.

Successful characterization and analysis of microbiomes depends on the ability to zoom in on these communities and identify the individual species and strains living within them. To date, most techniques for identifying the individual species within a microbiome provide insufficient resolution. Existing methods are also not effective in the characterization of an important class of genetic materials that can shuttle between different bacterial species, known as mobile genetic elements. Current characterization methods rely on having some knowledge or expectation about the composition of the microbial community and have thus far been largely performed in synthetic, cultured microbiome samples. Unfortunately, cultured microorganisms represent only a small fraction of natural microbial communities and hence the microbial diversity in terms of species richness and species abundance is grossly underestimated. As such, there is a need for more advanced technologies to effectively resolve high-complexity microbiome communities, such as environmental microbiomes and human gut microbiomes.

### SUMMARY OF THE INVENTION

The present disclosure is based, at least in part, on the identification of computer-implemented methods for deconvoluting metagenomic assembled contigs from a microbiome sample using nanopore sequencing. The methods disclosed herein were shown to be capable of de novo discovery and characterization chemical modifications within microbiome samples collected from bacterial and mammalian sources. Accordingly, the computer-implemented methods are effective in resolving high-complexity microbiomes for therapeutic, diagnostic, and environmental purposes.

Accordingly, the present disclosure provides, in some aspect, computer-implemented methods for deconvoluting metagenomic assembled contigs from a microbiome. In some embodiments, computer-implemented methods disclosed herein may include the following steps 9a) extracting DNA from the microbiome sample; (b) subjecting the extracted DNA to a single-molecule sequencing reaction using single-molecule sequencing technology to generate a raw signal; (c) processing the raw signal; (d) comparing the processed raw signal and a known raw signal, wherein the known raw signal is generated from a biomolecule consisting of matched sequence; (e) computing DNA modification feature vectors from deviation between processed raw signal and the known raw signal for at least one sequence motif in at least two metagenomic assembled contigs; (f) selecting DNA modification features predicting a DNA modification within the sequence motifs in at least one of the metagenomic assembled contigs; and (g) binning metagenomic assembled contigs according to similarity of DNA modification profile matrix into clusters. In some embodiments, computer-implemented methods may process raw signal by optionally mapping the raw signal to a known sequence of canonical monomers followed by reinforcing the raw signal. In some examples, methods of reinforcing raw signal can be accomplished by at least one method selected from the group of normalization, filtering, outlier removal, and aggregation. In some examples, a DNA modification can include at least one DNA modification type selected from the group of methylation, hydroxymethylation, phosphorothioates, glucosylation and hexosylation. In some other examples, DNA modification feature vectors computed from deviation between processed raw signal and the known raw signal for at least one sequence motif in at least two metagenomic assembled contigs can include at least of length two.

In some embodiments, computer-implemented methods herein that select for DNA modification features by predicting a DNA modification within the sequence motifs in at least one of the metagenomic assembled contigs, can do so by optionally determining a filtering criteria wherein the filtering criteria comprises at least one criterion selected from the group of feature value, feature frequency within metagenomic assembled contig, metagenomic assembled contig length, metagenomic assembled contig coverage, or sequence motif length.

In some other embodiments, computer-implemented methods herein that bin metagenomic assembled contigs according to similarity of DNA modification profile matrix into clusters can do so by optionally creating a DNA modification profile matrix comprised of at least one DNA modification feature vector for at least one sequence motif for at least two contigs.

In some embodiments, computer-implemented methods herein that subjecting the extracted DNA to a single-molecule sequencing reaction using single-molecule sequencing technology to generate a raw signal can do so by optionally subjecting the extracted DNA to a single-molecule sequencing reaction using nanopore sequencing technology to generate a raw signal.

In some embodiments, computer-implemented methods herein can use deconvolution of metagenomic contigs from a microbiome sample to match at least one mobile genetic element to at least one host genome. In some examples, the mobile genetic element can be a plasmid, a transposon, or a bacteriophage. In other examples, the mobile genetic element can include at least one sequence motif of interest.

In some embodiments, computer-implemented methods herein can use deconvolution of metagenomic contigs from a microbiome sample to diagnose, treat, classify, or a combination thereof at least one disease. In some embodiments, computer-implemented methods herein can use deconvolution of metagenomic contigs from a microbiome sample to determine at least one contamination of location of microbiome sample collection. In some examples, the microbiome sample can include at least two genomes of individual microorganisms. In some examples, the microbiome sample may be at least one source. In some examples, the microbiome sample source may be a protozoa, an animal, a human, or a plant. In some other examples, the microbiome sample source may be soil, air, water, sediment, oil, or combinations thereof.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A and 1B include diagrams depicting schematics for method design and applications. FIG. 1A: Shows a broadly applicable method using isolated bacteria with a wide variety of methylation motifs to explore signals of DNA methylation in nanopore sequencing and characterize the major types of DNA methylation (4mC, 5mC, and 6 mA), classifying DNA methylation into specific methylation type (4mC, 5mC, and 6 mA), and fine mapping of methylated bases. FIG. B: Shows an application of the disclosed method for methylation discovery from individual bacterial species and microbiome (methylation motif detection, classification, and fine mapping), as well as methylation-assisted metagenomic analysis (methylation binning and misassembly identification).

FIGS. 2A-2C include diagrams depicting systematic examination of three main types of DNA methylation with nanopore sequencing. FIG. 2A: Shows variation of current differences across methylation occurrences as illustrated by motif signatures from three motifs (AG4mCT (top panel), GGW5mCC (middle panel), and GCYYG6mAT (bottom panel)). For each motif, current differences near methylated bases ([−6 bp, +7 bp]) from all isolated occurrences were plotted with conservation of relative distances to methylated bases. Distributions of current differences for each relative distance are displayed as violin plots. Current differences axis shown is limited to −8 to 8 pA range. FIG. 2B: Shows variation of current differences across methylation occurrences as illustrated by projection with t-SNE from for 46 well-characterized motifs described in Table 2 herein. Each dot represents one isolated motif occurrence colored by methylation motif. For each motif occurrence, current differences from 22 positions near methylated bases ([−10 bp, +11 bp]) were used. A region showing multiple motifs with the same methylation type (see c) having similar signal is highlighted. FIG. 2C: Shows variation of current differences across methylation occurrences, similar to FIG. 2B but colored by DNA methylation type with additional processing to reveal cluster density indicated by relief.

FIGS. 3A-3C include diagrams depicting local sequence context effect on motif signature sand sequence-dependent variation in current differences for GGW5mCC methylation motif occurrences. FIG. 3A: Shows current differences from the violin plots of GGW5mCC in FIG. 2A plotted as a heatmap with each row representing current differences flanking a methylation occurrence ([−5, +6] relative to methylation). GGW5mCC motif occurrences were split into two groups according to degenerated base (W=[A|T] where “A” is the top panel and “T” is the bottom panel) and ordered, within groups, using hierarchical clustering to highlight current difference patterns. FIG. 3B: Shows t-SNE projection of motif occurrences from FIG. 3A with cluster density displayed as relief. Clusters are colored according to degenerated bases. FIG. 3C: Shows another example of sequence-dependent variation for GAT5mC motif occurrences with cluster density displayed as relief. Clusters are colored according to the first base following GAT5mC motif.

FIGS. 4A-4D include diagrams depicting the classification and fine mapping of three types of DNA methylation. FIG. 4A: Shows a schematic representation of dataset building for classifier training. For each motif occurrence, 7 training vectors of length 12 with +/− offsets from 0 to 3 position(s) relative to current differences core defined as [−2, +3] were produced. FIG. 4B: Shows each training vector labeled with the corresponding methylation type and offset used herein. The training vectors were then gathered into a large training dataset of current differences flanking 183,707 methylated bases from 45 distinct motifs. This dataset of current differences near the methylated base was used to train classifiers. FIG. 4C: Shows how classifiers' performances were evaluated using leave one out cross validation (LOOCV). FIG. 4D: Shows a subset of classifier evaluation results. Nine models were trained for each holdout combination to evaluate their performance for classifying holdout motifs. Every individual occurrence of each holdout motif and computed percentage of occurrences for each of the 21 labels using each classifier was performed separately. Results for six selected motifs are shown. Within motif predictions are displayed. Filling colors correspond to percentage of occurrences classified to a specific class ranging from blue (0%) to red (100%). Blank columns correspond to within-motif positions without prediction. Prediction percentages of expected classes are displayed in italic and fine mapped methylated positions in each motif are displayed in bold.

FIGS. 5A-5C include diagrams depicting a methylation analysis of mouse gut microbiome sample. FIG. 5A: Shows automated methylation binning of mouse gut microbiome metagenome contigs (without precise methylation motif discovery). Methylation status of common motifs (n=210,176) was screened across large contigs (>=500 kb) through computation of methylation feature vector. Informative motifs were selected and their status evaluated across remaining contigs. Resulting methylation features are projected on two dimensions using t-SNE. Contigs are colored based on bin identities assigned previously from the SMRT study with point sizes matching contig length according to legend. Discovered bins were manually defined based on clustering. Contigs marked with an asterisk were used as example for misassembly detection in FIG. 5C. FIG. 5B: Shows methylation-based association of MGEs to host genomes. Annotation of potential MGEs was obtained previously from the SMRT study. Genomic contigs are colored by bin of origin with point sizes matching their length. FIG. 5C: Detection of misassemblies using methylation motif information along contigs. The top two panels: misassembled contigs mislabeled as Bin 7 in SMRT analysis (PDYJ01003082.1 (top panel) and PDYJ01003083.1 (middle panel) contigs marked with an asterisk in FIG. 5A. Bottom panel depicts a properly assembled contig from Bin 7 (PDYJ01000763.1). Some de novo detected motifs from Bin 7 were selected, and their methylation sites were scored along the three contigs. Methylation scores were then smoothed using locally estimated scatterplot smoothing and displayed with one color per motif. Smoothed methylation scores are consistent in contig from bottom panel, but not in the misassembled contigs shown in the top two panels. A switch of methylome occurs near 800 kbp and 300 kb respectively, supporting the existence of misassemblies.

FIGS. 6A-6C include diagrams depicting general statistics of motif signatures. FIG. 6A: Distribution of current differences are shown for all confident motifs altogether (left panel) as well as average absolute differences (right panel) and associated standard deviations near methylated bases ([−10, +11]). FIG. 6B: Shows distribution of current differences in a manner similar to FIG. 6A with a distinction between the DNA methylation types 4mC (top panel), 5mC (middle panel), and 6 mA (bottom panel). FIG. 6C: Shows distribution of current differences in a manner similar to FIG. 6A but for individual methylation motifs.

FIGS. 7A and 7B include diagrams depicting systematic examination of three main DNA methylation types with nanopore sequencing. FIG. 7A: Shows a t-SNE projection of isolated methylation motif occurrences separated per motif. The same dataset as FIG. 2B was used with occurrences colored per motif. FIG. 7B: Shows a t-SNE projection of isolated methylation motif occurrences separated per motif like FIG. 7A, but grouped by methylation type.

FIGS. 8A-8D include diagrams depicting additional information for classification of methylation motif occurrences. FIG. 8A: Shows an approximation of DNA methylation position in three motifs (AGCT (left panels), GCYYGAT (middle panels), and GGWCC (right panels)). Signal strength was computed using a sliding window alongside motif signature to choose the best vector positioning to use for classification. FIG. 8B: Shows a flowchart description of procedure for classifier training and novel motifs dataset annotation. FIG. 8C: Shows a boxplot of overall prediction accuracy in LOOCV evaluation for each classifier. Classifiers were ordered by average accuracy. FIG. 8D: Shows the effect of hyperparameters on classification accuracy. Boxplot of overall prediction accuracy in LOOCV evaluation with classifiers trained on all motifs except the ones from H. pylori. Hyperparameters were either tuned on H. pylori motifs only (“Alt. HP”) or on all motifs (“Main HP”).

FIG. 9 includes diagrams depicting classification and fine mapping of three types of DNA methylation (part 1) similar to FIG. 4B with full set of prediction results for a subset of methylation motifs. Filling colors correspond to percentage of occurrences classified to a specific class ranging from blue (0%) to red (100%). Greyed out prediction correspond to out of motif position. Blank columns correspond to within-motif positions without prediction. Prediction percentages of expected classes are displayed in italic and chosen one based on consensus are displayed in bold.

FIG. 10 includes diagrams depicting classification and fine mapping of three types of DNA methylation (part 2) similar to FIG. 4B with full set of prediction results for a subset of methylation motifs. Filling colors correspond to percentage of occurrences classified to a specific class ranging from blue (0%) to red (100%). Greyed out prediction correspond to out of motif position. Blank columns correspond to within-motif positions without prediction. Prediction percentages of expected classes are displayed in italic and chosen one based on consensus are displayed in bold.

FIGS. 11A and 11B include diagrams depicting an evaluation of motif enrichment with Precision-Recall curves. FIG. 11A: Shows an effect of coverage on de novo methylated site detection. Individual motif occurrences detection was evaluated using Precision-Recall curves (PR curves) for H. pylori. Studied datasets with coverage ranging from 5× to 200× were generated by random subsampling of native and WGA datasets. Precision-Recall curves were generated as described herein where only confident H. pylori motifs were considered for evaluation. FIG. 11B: Shows precision-Recall curves summarizing the detection performance at 75× coverage of individual methylation sites for each motif in H. pylori with adjusted frequency.

FIG. 12 includes a diagram depicting a schematic representation of methylation feature vectors computation and methylation binning of contigs.

FIG. 13 includes diagrams depicting detection of misassemblies in Bin 7 contigs from methylation motif signal. Identification of contamination origin for the two contigs mislabeled as Bin 7 (PDYJ01003082.1 (left panels) and PDYJ01003083.1 (right panels), marked with an asterisk in FIG. 5A). Occurrences from methylation motifs found in each bin were scored separately and smoothed signal along misassembled contigs. Scores from motif occurrences overlapping Bin 7 motifs were removed. Scores from Bin 2 motifs are consistently high in the second half of contig PDYJ01003082.1 and first half of contig PDYJ01003083.1 suggesting contamination originated from Bin 2 genomic sequences.

FIG. 14 includes a diagram depicting a motif signature for CC6mACC in N. gonorrhoeae. Current differences axis was limited to −8 to 8 pA range.

### DETAILED DESCRIPTION

Newer sequencing methods (e.g. nanopore sequencing) provide a great opportunity for direct detection of chemical DNA modification. However, currently used computational methods that assess the detected chemical DNA modifications have been trained to detect a specific form of DNA modification from one, or few, specific sequence contexts (e.g. 5-methylcytosine from CpG dinucleotides). The present disclosure is based, at least in part, on the surprising discovery that nanopore sequencing signal displays showed complex heterogeneity, even across methylation events of the same type. This observation implied that nanopore sequencing based detection of DNA modifications is best developed using datasets gathered from a broad collection of sequence contexts in order to be broadly applicable for modification discovery. Accordingly, the methods disclosed herein use training datasets from a diverse assortment of bacterial species to develop a novel classification method for identifying and fine mapping of DNA modifications. Additionally, the methods disclosed herein can be used to analyze complex metagenomes within microbiome samples.

The present disclosure provides computer-implemented methods for deconvoluting metagenomic assembled contigs from a microbiome sample. In general, the methods disclosed herein subject a microbiome sample to a single-molecule sequencing reaction, process resulting sequence data, compute DNA modification features, selecting DNA modification features predicting a DNA modification within the sequence motifs in at least one of the metagenomic assembled contigs, and binning metagenomic assembled contigs according to similarity of DNA modification profile matrix into clusters. Various embodiments of the disclosure are described in more detail below.

Described herein are several definitions. Such definitions are meant to encompass grammatical equivalents.

The term “biomolecule” is intended to be a generic term, which includes for example (but not limited to) nucleic acids. In some aspects, a biomolecule is DNA.

The term “microbiome” refers to either the collective genomes of prokaryotic organisms that reside in an environmental niche or the collective genomes microorganisms themselves. A microbiome may include collective genomes of prokaryotic organisms selected from bacteria, archaea, protists, fungi, viruses, or a combination thereof.

The term “contig” refers to a set of overlapping DNA segments that together represent a consensus region of DNA.

The term “match sequence” refers to a level of sequence similarity equivalent to a BLAST score ranging from 40 (the equivalent of 20 consecutive identical nucleotides/amino acids) to 2000 (the equivalent of 1000 consecutive identical nucleotides/amino acids).

“BLAST” (Basic Local Alignment Search Tool) is a technique for detecting ungapped sub-sequences that match a given query sequence. BLAST is used in one embodiment of the present invention as a final step in detecting sequence matches.

“BLASTP” is a BLAST program that compares an amino acid query sequence against a protein sequence database.

“BLASTX” is a BLAST program that compares the six-frame conceptual translation products of a nucleotide query sequence (both strands) against a protein sequence database.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

Furthermore, the use of the term “including,” as well as other forms, such as “includes” and “included,” is not limiting. Also, terms such as “element” or “component” encompass both elements and components including one unit and elements and components that comprise more than one subunit unless specifically stated otherwise.

Any prokaryotic organisms known to those skilled in the art are within the scope of the present disclosure. In some non-limiting embodiments, prokaryotic organisms include bacterial organisms, archaeal organisms, and combinations thereof. In other non-limiting embodiments, prokaryotic organisms include bacterial organisms, bacterial species, or strains of bacterial species. In still other non-limiting embodiments, the prokaryotic organisms include archaeal organisms, archaeal species, or strains of archaeal species.

Microbiome samples analyzed by the methods disclosed herein can be obtained from any source known to those skilled in the art. In some non-limiting embodiments, a microbiome sample can be obtained from soil, air, water (including, without limitation, marine water, fresh water, and rain water), sediment, oil, and combinations thereof. In other non-limiting embodiments, a microbiome sample can be obtained from a subject selected from a protozoa, an animal (e.g., a mammal, e.g., human), or a plant.

The term “subject” as used herein refers to an animal, including but not limited to a mammal including a human and a non-human primate (for example, a monkey or great ape), a cow, a pig, a cat, a dog, a rat, a mouse, a horse, a goat, a rabbit, a sheep, a hamster, a guinea pig). Preferably, the subject is a human. In some embodiments, a subject is at a genetic risk for development a disease. Non-limiting examples of such diseases include digestive system diseases, cardiovascular diseases, neurological diseases, obesity, diabetes, and cancers. In other embodiments, the subject may be at a risk of having, or have a bacterial infection, e.g., pneumonia infection.

In various embodiments, a sample obtained from an animal subject can be a body fluid. In other embodiments, a sample obtained from an animal subject can be a tissue sample. Non-limiting samples obtained from an animal subject include tooth, perspiration, fingernail, skin, hair, feces, urine, semen, mucus, saliva, and gastrointestinal tract samples. In some aspects, a human microbiome sample encompasses collection of microorganisms found on the surface and deep layers of skin, in mammary glands, saliva, oral mucosa, conjunctiva and gastrointestinal tract. In some aspects, microorganisms found in the microbiome can include bacteria, fungi, protozoa, viruses and archaea. In other aspects, different parts of a subject's body may exhibit varying diversity of microorganisms. In still other aspects, quantity and/or type of microorganisms may signal a healthy state or a diseased state of a subject whose microbiome was collected from. In yet other aspects, a bacterial composition for a given site on a subject's body may vary from subject to subject, not only in type, but also in abundance or quantity.

In some embodiments, the prokaryotic organisms in the microbiome sample do not have high sequence similarity. In some embodiments, two or more of the prokaryotic organisms in the microbiome sample have high sequence similarity. In some embodiments, two or more of the prokaryotic organisms in the microbiome sample have an average nucleotide identity of greater than about 75%, than about 80%, than about 85%, than about 90%, than about 95%, than about 97%, than about 98%, or than about 99%.

In various embodiments, mobile genetic elements of any size can be mapped using the methods disclosed herein. In some embodiments, the mobile genetic element is greater than about 1 kbp in length, or greater than about 2 kbp, or greater than about 5 kbp, or greater than about 10 kbp, or greater than about 20 kbp, or greater than about 30 kbp. In one non-limiting embodiment, the mobile genetic element is greater than 10 kbp in length.

In some embodiments, a mobile genetic element confers certain properties to the host subject. In another embodiment the mobile genetic element encodes a virulence factor in the prokaryotic host subject. In yet another embodiment the mobile genetic element provides a metabolic function to the prokaryotic host subject.

In various embodiments, microbiome samples of any size or complexity are within the scope to be analyzed by the methods disclosed herein. In one embodiment, a microbiome sample analyzed by methods disclosed herein may be greater than 1, or greater than 3, or greater than 5, or greater than 10, or greater than 20, or greater than 50, or greater than 75, or greater than 100, or greater than 200, or greater than 300, or greater than 400, or greater than 500, or greater than 700, or greater than 1000, or greater than 2000, or greater than 5000, or greater than 10,000 prokaryotic host organisms.

In various embodiments, a DNA modification may be methylation, hydroxymethylation, phosphorothioates, glucosylation, hexosylation, or combinations thereof. In preferred embodiments, the DNA modification may be methylation. Any methylated nucleotides are within the scope of the methods disclosed herein. In some aspects, the methylated nucleotides may be selected from, without limitation, N⁶-methyladenine, N⁴-methylcytosine, 5-methylcytosine and combinations thereof.

In various embodiments, microbiome samples for use with the methods provided herein can encompass, without limitation, samples obtained from the environment, including soil (e.g., rhizosphere), air, water (e.g., marine water, fresh water, rain water, wastewater sludge), sediment, oil, an extreme environmental sample (e.g., acid mine drainage, hydrothermal systems) and combinations thereof. In some aspects, marine or freshwater samples can be from the surface of the body of water, or any depth of the body of water, e.g., a deep sea sample. In other aspects, a water sample may be an ocean, a sea, a river, a lake, or a sewage sample. In still other aspects, a water sample can be sourced from a water-treatment facility, a sewage facility, or any building in need thereof.

In some embodiments, a computer-implemented method of deconvoluting metagenomic assembled contigs from a microbiome sample can encompass the following steps: (a) extracting DNA from the microbiome sample; (b) subjecting the extracted DNA to a single-molecule sequencing reaction using single-molecule sequencing technology to generate a raw signal; (c) processing the raw signal; (d) comparing the processed raw signal and a known raw signal, wherein the known raw signal is generated from a biomolecule consisting of matched sequence; (e) computing DNA modification feature vectors from deviation between processed raw signal and the known raw signal for at least one sequence motif in at least two metagenomic assembled contigs; (f) selecting DNA modification features predicting a DNA modification within the sequence motifs in at least one of the metagenomic assembled contigs; and (g) binning metagenomic assembled contigs according to similarity of DNA modification profile matrix into clusters. In some examples, methods herein may include at least one type of DNA modification. In some aspects, a DNA modification type can be selected from the group of methylation, hydroxymethylation, phosphorothioates, glucosylation and hexosylation.

In some embodiments, a computer-implemented methods herein that include processing of a raw signal can do so by (a) mapping the raw signal to a known sequence of canonical monomers; and (b) reinforcing the raw signal. In other embodiments, method of reinforcing raw signal as disclosed herein can be accomplished by at least one method selected from the group of normalization, filtering, outlier removal, and aggregation.

In some embodiments, a computer-implemented methods herein that include determining a filtering criteria, can do by at least one criterion. In some examples, a criterion used herein can be selected from the group of feature value, feature frequency within metagenomic assembled contig, metagenomic assembled contig length, metagenomic assembled contig coverage, or sequence motif length.

In some embodiments, a computer-implemented methods herein that include binning metagenomic assembled contigs according to similarity of DNA modification profile matrix into clusters may create a DNA modification profile matrix that includes at least one DNA modification feature vector for at least one sequence motif for at least two contigs. In some examples, the DNA modification feature vector computed can be about of length two to about of length 50. In exemplary examples, the DNA modification feature vector computed is at least of length two.

In some embodiments, microbiome samples for use in methods herein can be from one source to 10 sources. In exemplary examples, microbiome samples for use in methods herein can be from at least one source. In some aspects, sources may be selected from the group of a protozoa, an animal, a human or a plant. In other aspects, sources may be selected from the group of soil, air, water, sediment, oil, or combinations thereof. In some aspects where the sample source is water, a water source can be selected from the group of marine water, fresh water, and rainwater.

In some embodiments, a microbiome sample for use in methods herein can encompass at least two genomes to at least 20 genomes of individual microorganisms. In exemplary examples, a microbiome sample can encompass at least two genomes of individual microorganisms. In some aspects, microbiome samples as disclosed herein microorganisms can be at least one bacteria, archaea, fungi, protozoa, viruses, or combinations thereof. In other aspects, microorganisms can be species from same genus. In some other aspects, microorganisms can be strains from the same species.

In some embodiments, methods of subjecting extracted DNA to a single-molecule sequencing reaction using single-molecule sequencing technology to generate a raw signal can include subjecting the extracted DNA to a single-molecule sequencing reaction using nanopore sequencing technology to generate a raw signal.

In some embodiments, computer-implemented methods herein can use deconvolution of metagenomic contigs from the microbiome sample to match at least one mobile genetic element to at least one host genome, at least two mobile genetic elements to at least two host genomes, at least six mobile genetic elements to at least six host genomes, at least eight mobile genetic elements to at least eight host genomes, or at least ten mobile genetic elements to at least ten host genomes. In some examples, deconvolution of metagenomic contigs from the microbiome sample can be used to match unlimited mobile genetic elements to unlimited host genomes. In some preferred examples, deconvolution of metagenomic contigs from the microbiome sample can be used to match at least one mobile genetic element to at least one host genome. In some aspects, mobile genetic elements can include a plasmid, a transposon, or a bacteriophage. In other aspects, mobile genetic elements can include at least one to at least 50 sequences motif of interest. In some preferred aspects, mobile genetic elements can include at least one sequences motif of interest.

In some examples, mobile genetic elements disclosed herein may confer antibiotic resistance to the host microorganism. In some examples, mobile genetic elements disclosed herein may encode at least one virulence factor in the host microorganism. In some aspects, mobile genetic element can provide at least one metabolic function to the host microorganism.

In some embodiments, computer-implemented methods herein can use deconvolution of metagenomic contigs to diagnose at least one disease. In some embodiments, computer-implemented methods herein can use deconvolution of metagenomic contigs to determine resistance to at least one antibiotic. In some embodiments, computer-implemented methods herein can use deconvolution of metagenomic contigs to determine at least one contamination of location of microbiome sample collection.

In some embodiments, computer-implemented methods of deconvoluting metagenomic single molecule reads from a microbiome sample herein, can optionally include a step of computing DNA modification feature vectors from deviation between processed raw signal and the known raw signal for at least one sequence motif in at least two single molecule reads.

In some embodiments, computer-implemented methods of deconvoluting metagenomic single molecule reads from a microbiome sample herein, can optionally include a step of creating a DNA modification profile matrix comprised of at least one DNA modification feature vector for at least one sequence motif for at least two single molecule reads.

In some embodiments, computer-implemented methods of improving deconvolution metagenomic assembled contigs from a microbiome sample can encompass the following steps: (a) extracting DNA from the microbiome sample; (b) subjecting the extracted DNA to a single-molecule sequencing reaction using single-molecule sequencing technology to generate a raw signal; (c) processing the raw signal; (d) detecting differences between the processed raw signal and a known raw signal, wherein the differences indicate chemical modifications in close proximity, and the known raw signal is generated from a biomolecule consisting of matched sequence; (e) identifying sequence motifs associated with de novo detected DNA modifications in at least one metagenomic assembled contig cluster; (f) computing DNA modification feature vectors from deviation between processed raw signal and the known raw signal for at least one sequence motif in at least two metagenomic assembled contigs; and (g) binning the metagenomic assembled contigs according to similarity of DNA modification profile matrix into clusters. In some aspects, methods herein that include binning the metagenomic assembled contigs according to similarity of DNA modification profile matrix into clusters may create a DNA modification profile matrix comprised of at least one DNA modification feature vector for at least one sequence motif for at least two metagenomic assembled contigs.

In some embodiments of methods disclosed herein, detection of differences between a processed raw signal and a known raw signal may indicate chemical modifications in close proximity, and the known raw signal is generated from a biomolecule consisting of matched sequence.

In some embodiments of methods disclosed herein, computing DNA modification feature vectors can be performed from deviation between processed raw signal and the known raw signal for at least one sequence motif in at least two metagenomic single molecule reads.

In some embodiments, computer-implemented methods of detecting abnormal changes in DNA modification status that can indicate erroneous contig in a metagenome assembly from microbiome sample using similarity of methylation profile, can encompass the following steps: (a) extracting DNA from the microbiome sample; (b) subjecting the extracted DNA to a single-molecule sequencing reaction using single-molecule sequencing technology to generate a raw signal; (c) processing the raw signal; (d) comparing the processed raw signal and a known raw signal, wherein the known raw signal is generated from a biomolecule consisting of matched sequence; (e) computing a score for at least two occurrences of a sequence motif of interest in a metagenomic assembled contig; wherein the computed score reflect the DNA modification status of an occurrence of the sequence motif; (f) generating a map of DNA modification status of at least one sequence motif of interest in a metagenomic assembled contig of the microbiome sample; and (g) identifying abnormal changes in DNA modification status from at least one sequence motif along the metagenomic assembled contig.

In some embodiments, methods herein that subject extracted DNA to a single-molecule sequencing reaction using single-molecule sequencing technology to generate a raw signal may use at least nanopore sequencing technology. In some aspects, a nanopore sequencing technology may be Single Molecule Real-Time (SMRT) sequencing to generate a raw signal.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. Nature of Nanopore Sequencing Signal from Oxford Nanopore Technologies

Raw nanopore signal corresponds to electric current level (pA) sampled at 4000 hz across the nanopore while a DNA strand is transferred from one compartment to the other in a 450 bp.s-1 ratcheting motion. Higher order of signal structure, called events, consists in consecutive signal level corresponding to multiple measures of current for a specific relative position of the DNA strand inside the pore. The initial signal processing performed by the base caller, Albacore (version 1.1.0), detects those consecutive events and translates them into a nucleotide sequence.

### Example 2. Heterogeneous Signal Variation Induced by DNA Methylation in Nanopore Sequencing

In the bacterial kingdom, DNA methylation has three primary forms: 6 mA, 4mC and 5mC, all of which occur in a highly motif-driven manner: on average, each bacterial genome contains three methylation motifs, and nearly every occurrence of the target motifs is methylated. While 6 mA motifs are most prevalent in bacteria, 4mC and 5mC motifs are less common. In order to comprehensively examine the variation of different types of DNA methylation within a broad scope of sequence context as measured by nanopore sequencing, we collected 46 well-characterized unique methylation motifs were collected from a set of bacterial species with diverse methylation motifs (Table 1).

TABLE 1
List of bacterial strains analyzed

| | Genome | Reference | |
|---|---|---|---|
| Organism name | size (Mbp) | genome/Assembly used | REBASE annotation |
| Bacillus amyloliquefaciens H | 3.95 | Not released yet | Not released yet |
| Bacillus fusiformis 122 | 4.97 | Not released yet | Not released yet |
| Clostridium perfringens ATCC | 3.26 | https://www.ncbi.nlm.nih. | http://rebase.neb.com/cgi- |
| 13124 | | gov/nuccore/NC 008261.1 | bin/pacbioget?4467 |
| Escherichia coli K-12 substr. | 4.66 | https://www.ncbi.nlm.nih.g | http://rebase.neb.com/cgi- |
| MG1655 | | ov/nuccore/CP014225.1 | bin/pacbioget?17068 |
| Helicobacter pylori JP26 | 1.58 | Not released yet | Not released yet |
| Methanospirillum hungatei JF-1 | 3.54 | https://www.ncbi.nlm.nih. | http://rebase.neb.com/cgi- |
| | | gov/nuccore/NC 007796.1 | bin/pacbioget?4278 |
| Neisseria gonorrhoeae FA 1090 | 2.15 | https://www.ncbi.nlm.nih. | http://rebase.neb.com/cgi- |
| | | gov/nuccore/NC 002946.2 | bin/pacbioget?1851 |

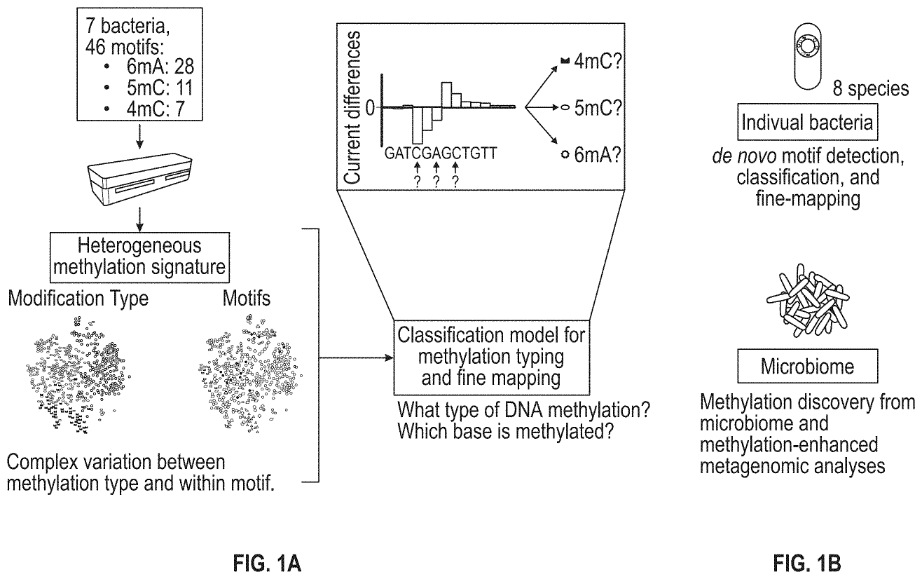

According a REBASE curated database, these strains have a total of 46 unique and confident methylation motifs covering the three major methylation types (6 mA motifs: 28; 4mC motifs: 7; 5mC motifs: 11; 308,773 methylation sites in total (FIGS. 1A and 1B; Table 2).

TABLE 2
List of confident motifs considered in motif detection analysis.
Number of motif occurrences across reference genome (both strands).

| | Methylation | Methylation | Motif | Number of | |
|---|---|---|---|---|---|
| Motif | type | position | length | Occurrences | Organism Name |
| G5mCWGC | 5mC | 2 | 5 | 23726 | Bacillus amyloliquefaciens H |
| GGAT4mCC | 4mC | 5 | 6 | 462 | |
| GAT5mC | 5mC | 4 | 4 | 18428 | Bacillus fusiformis 122 |
| 5mCCGG | 5mC | 1 | 4 | 780 | Clostridium perfringens ATCC 13124 |
| C6mACNNNNNRTAAA | 6mA | 2 | 13 | 279 | |
| GAT5mC | 5mC | 4 | 4 | 8520 | |
| GGW5mCC | 5mC | 4 | 5 | 2252 | |
| GTAT6mAC | 6mA | 5 | 6 | 318 | |
| TTT6mAYNNNNNGTG | 6mA | 4 | 13 | 279 | |
| VGAC6mAT | 6mA | 5 | 6 | 2122 | |
| A6mACNNNNNNGTGC | 6mA | 2 | 13 | 597 | Escherichia coli K-12 substr. MG1655 |
| C5mCWGG | 5mC | 2 | 5 | 24188 | |
| G6mATC | 6mA | 2 | 4 | 38368 | |
| GC6mACNNNNNNGTT | 6mA | 3 | 13 | 597 | |
| 4mCCGG | 4mC | 1 | 4 | 3422 | Helicobacter pylori JP26 |
| ATTA6mAT | 6mA | 5 | 6 | 876 | |
| C6mATG | 6mA | 2 | 4 | 14318 | |
| CRT6mANNNNNNNWC | 6mA | 4 | 13 | 1253 | |
| CS6mAG | 6mA | 3 | 4 | 8220 | |
| CTRY6mAG | 6mA | 5 | 6 | 1282 | |
| CY6mANNNNNNTTC | 6mA | 3 | 12 | 1056 | |
| G5mCGC | 5mC | 2 | 4 | 12072 | |
| G6mAGG | 6mA | 2 | 4 | 4583 | |
| G6mANNNNNNNTAYG | 6mA | 2 | 13 | 648 | |
| GA6mANNNNNNTRG | 6mA | 3 | 12 | 1056 | |
| GA6mATTC | 6mA | 3 | 6 | 298 | |
| GG5mCC | 5mC | 3 | 4 | 2918 | |
| GMRG6mA | 6mA | 5 | 5 | 7695 | |
| GT6mAC | 6mA | 3 | 4 | 198 | |
| GTNN6mAC | 6mA | 5 | 6 | 540 | |
| T4mCTTC | 4mC | 2 | 5 | 4555 | |
| TCG6mA | 6mA | 4 | 4 | 562 | |
| TCNNG6mA | 6mA | 6 | 6 | 3864 | |
| TGC6mA | 6mA | 4 | 4 | 11256 | |
| 4mCTNAG | 4mC | 1 | 5 | 10908 | Methanospirillum hungatei JF-1 |
| AG4mCT | 4mC | 3 | 4 | 11534 | |
| CCA4mCGK | 4mC | 4 | 6 | 1396 | |
| G6mATC | 6mA | 2 | 4 | 44388 | |
| GCYYG6mAT | 6mA | 6 | 7 | 2024 | |
| GTA4mC | 4mC | 4 | 4 | 15396 | |
| C5mCGCGG | 5mC | 2 | 6 | 438 | Neisseria gonorrhoeae FA 1090 |
| G5mCCGGC | 5mC | 2 | 6 | 3174 | |
| G6mAGNNNNNTAC | 6mA | 2 | 11 | 203 | |
| GC6mANNNNNNNNTGC | 6mA | 3 | 14 | 1832 | |
| GG5mCC | 5mC | 3 | 4 | 9190 | |
| GGNN5mCC | 5mC | 5 | 6 | 3762 | |
| GGTG6mA | 6mA | 5 | 5 | 1809 | |
| GT6mANNNNNCTC | 6mA | 3 | 11 | 203 | |
| RG5mCGCY | 5mC | 3 | 6 | 928 | |

Nanopore sequencing was conducted on MinION with R9.4 flow cells achieving 175× coverage on average (Table 3) for both the native DNA samples and their WGA samples. Read subsampling was used to allow systematic methods evaluation.

TABLE 3
Nanopore sequencing dataset coverage used for motif
detection and classification. Average coverages were computed
using bedtools (version 2.26.0, parameters genomecov -d).

| | | | Independent |
|---|---|---|---|
| Organism Name | Native | WGA | WGA |
| Bacillus amyloliquefaciens H | 186 | 119 | — |
| Bacillus fusiformis 122 | 154 | 102 | — |
| Clostridium perfringens ATCC 13124 | 250 | 129 | — |
| Escherichia coli K-12 substr. MG1655 | 200 | 200 | — |
| Helicobacter pylori JP26 | 200 | 200 | 200 |
| Methanospirillum hungatei JF-1 | 232 | 113 | — |
| Neisseria gonorrhoeae FA 1090 | 195 | 169 | — |

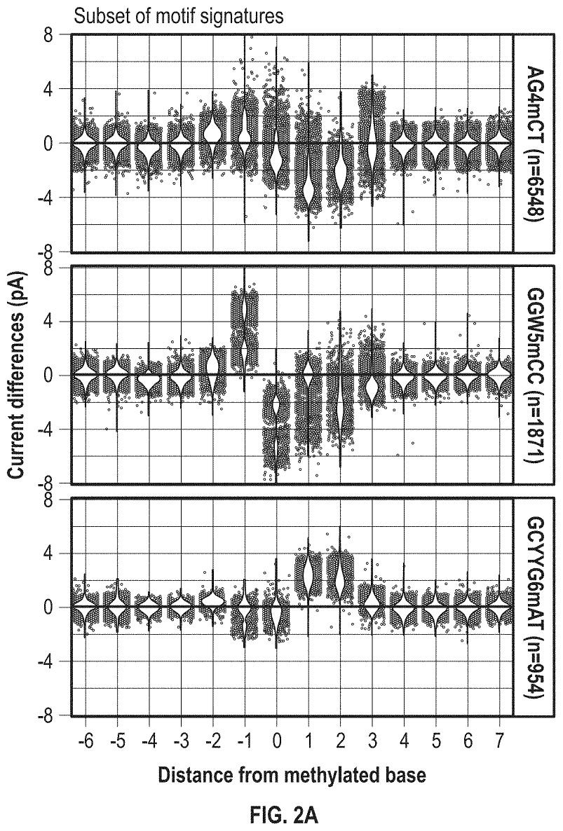

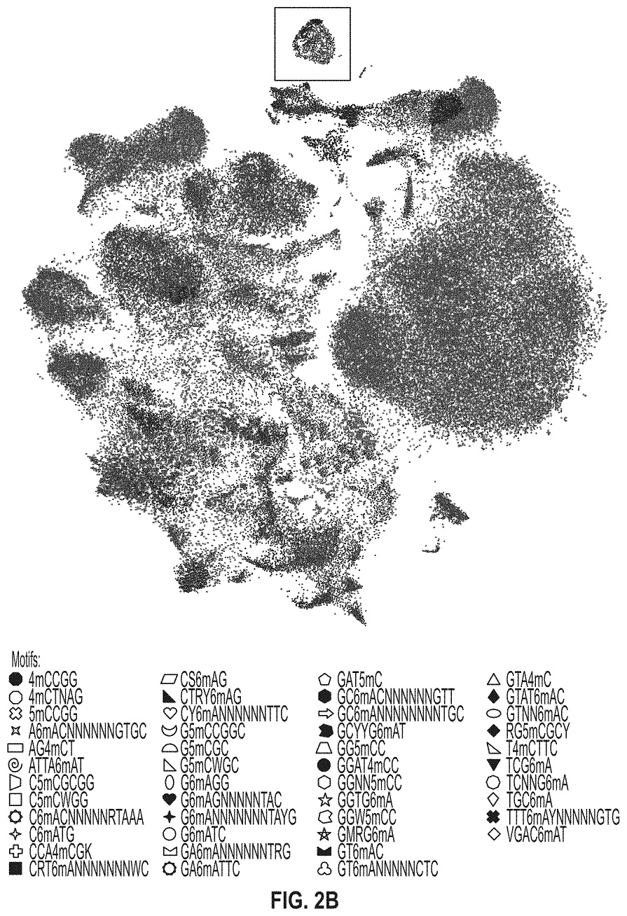

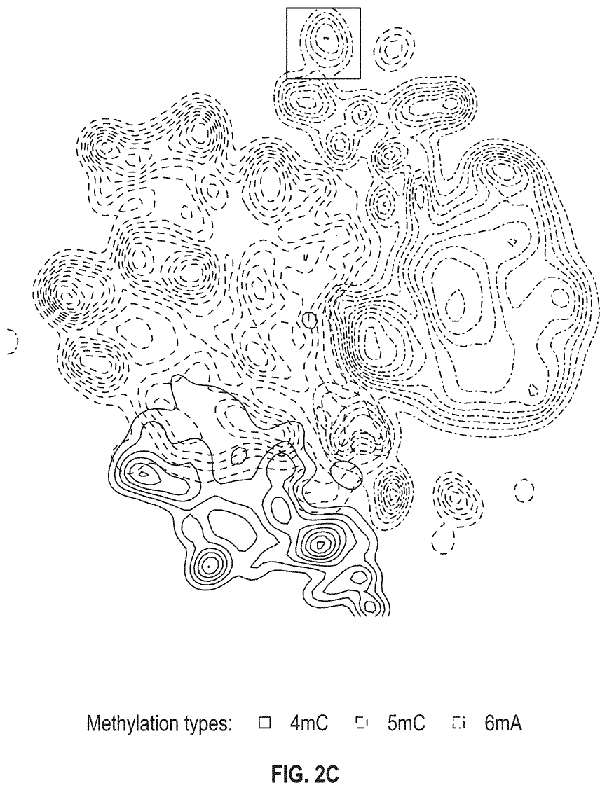

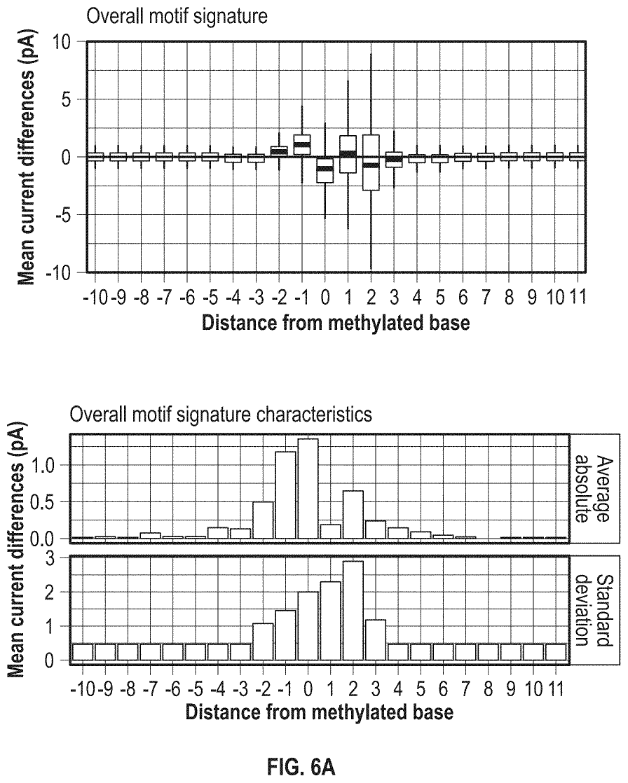

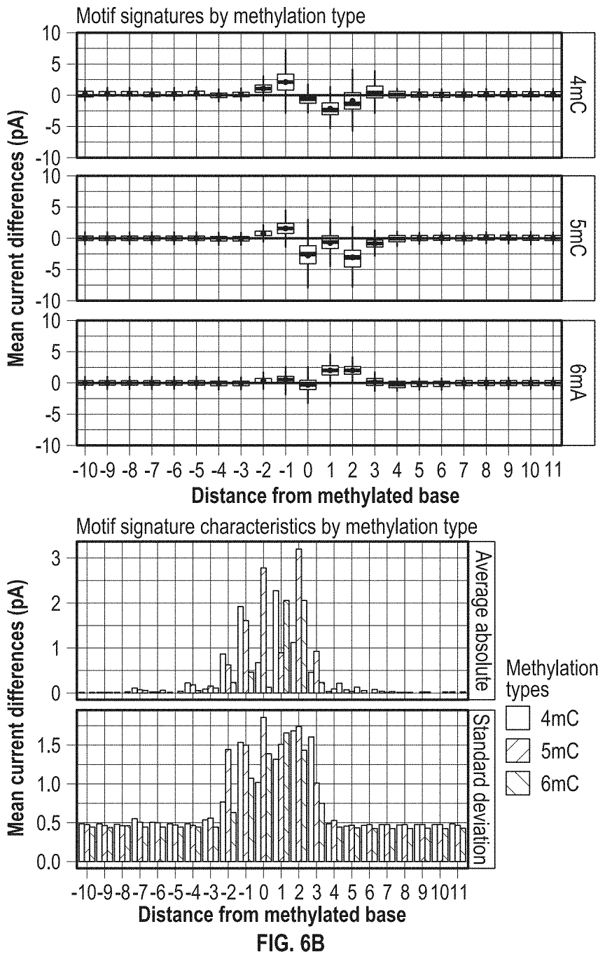

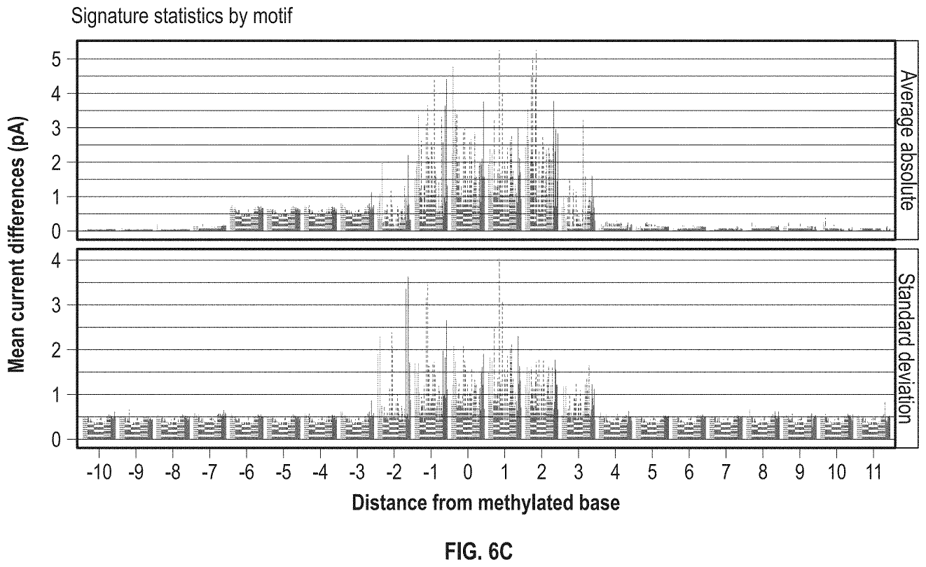

Read events and associated current levels (picoampere, pA) were aligned to reference genomes using Nanopolish. After normalization and filtering, current differences between native and WGA datasets were computed for each genomic position. To examine the variation of current differences across different DNA methylation types and motifs, we extracted current differences around each methylated base ([−6 bp, +7 bp]) and grouped them by methylation motifs. To avoid potential compound effect in the evaluation, methylation sites in the vicinity of each other were excluded. By superposing those current differences centered on the methylated base from every occurrence of a methylation motif, referred to as the methylation motif signature, we can study how current differences are affected by DNA methylation on average (FIG. 2A). Generally, the widths and amplitudes of perturbation in the methylation motif signatures vary between different motifs and methylation types (FIGS. 6A-6C). The broadness of signal perturbation suggests that methylation induces current differences across multiple flanking bases, essentially due to DNA methylation disturbing the ionic current of multiple consecutive events while ratcheting through the nanopore. It is worth noting that this broadness contrasts with the deviations of kinetic DNA polymerase confined to a single base for 4mC and 6 mA in SMRT sequencing.

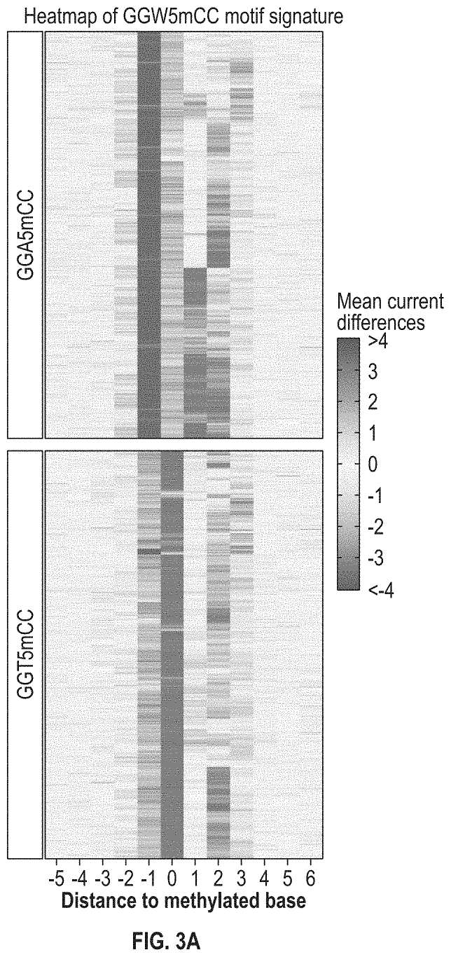

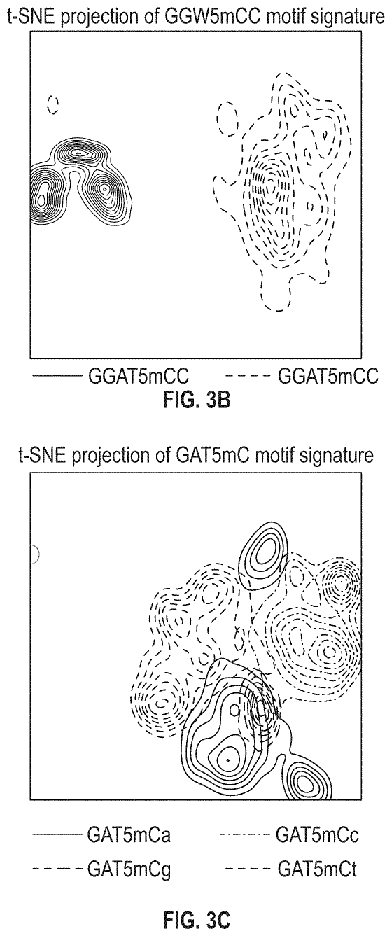

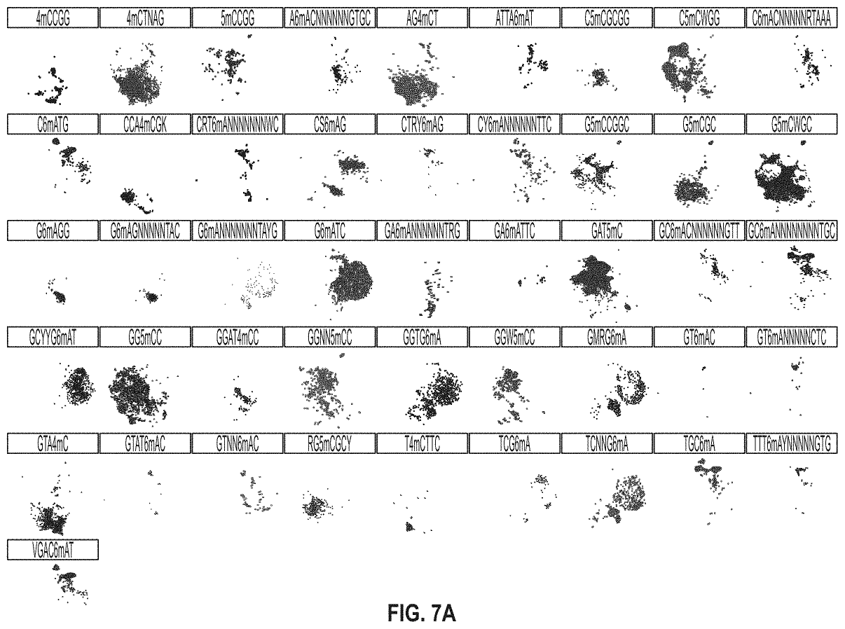

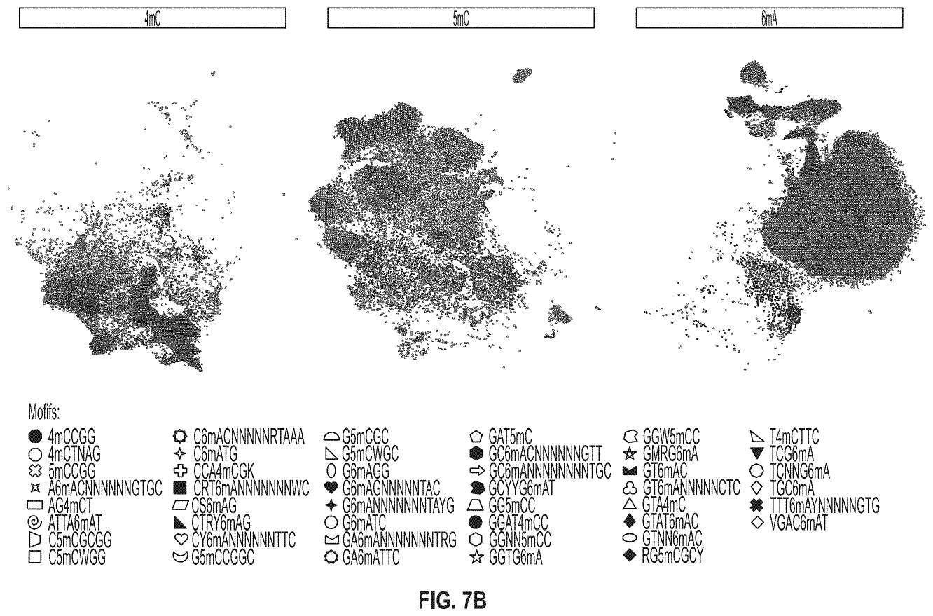

To obtain an overall view of the current differences across all the methylation types and methylation motifs, we subjected the 14 bp vectors ([−6 bp, +7 bp]) capturing current differences across 183,763 non-overlapping methylation motif occurrences to t-distributed stochastic neighbor embedding (t-SNE) a nonlinear dimensionality reduction algorithm (FIGS. 2B and 2C; FIGS. 7A and 7B). There is a general clustering pattern where methylation motif occurrences from the same methylation type tend to cluster together (FIG. 2C and FIG. 7B), although there are apparent overlaps. Importantly, we observed that current differences associated with different methylation motifs of the same methylation type often form different clusters, and some motifs even form distinct sub-clusters, i.e. current differences generally varies between different motifs of the same methylation type (FIG. 2C and FIG. 7B), and even between methylation events within the same methylation motif (FIGS. 2A and 2B; FIG. 7A). Further analysis of signatures for subsets of the same motif suggests that this across-motif and within-motif variation is due to sequence variation from degenerated position in motifs as well as sequences flanking the consensus motifs. In FIGS. 3A and 3B, we showed an illustrative example where signature sub-clusters for a 5mC motif (GGW5mCC) can be partially explained by sequence diversity near methylated bases (within-motif sequence variation). Similar observations were made with respect to sequence variation outside of consensus methylation motif (FIG. 3C).

In summary, these analyses showed that current differences induced by DNA methylation of the same type have great variation and heterogeneity in nanopore sequencing. This observation has important implications on methods development for nanopore sequencing based detection of DNA methylation. Specifically, it suggests that a broadly applicable method for methylation discovery is best trained using a comprehensive dataset with methylation motif diversity rather than a dataset of one or few specific motifs. This motivated us to develop the novel method that we will describe in the next section.

### Example 3. De Novo Identification of Methylation Type and Methylated Base

To account for the great signature diversity of methylation induced current differences across sequence contexts, we developed a novel method for the following two challenging tasks unaddressed yet by existing methods: 1) methylation type classification, where the goal is to identify the type of DNA methylation, and 2) fine mapping, where the goal is to identify the position of the methylated base.

Methylation motif enrichment. Before introducing the novel classification method, we need to first describe the procedure we used for methylation detection and motif enrichment analysis building on existing methods. In brief, 1) current levels are compared between native and WGA datasets for each genomic position; 2) p-values are combined locally with a sliding window-based approach followed by peak detection; 3) flanking sequences around the center of peaks are used as input for MEME motif discovery analysis. Overall, 45 of the total 46 well-characterized methylation motifs from seven bacteria were successfully re-discovered (Table 2). The only undetected motif, GT6mAC from H. pylori, has much fewer occurrences (i.e. only 198 in the entire genome) than other 4-mer motifs (7169 occurrences on average). The motif discovery analysis also revealed six additional motifs not among the 46 well-characterized motifs. One is likely a 5mC motif that was missed by SMRT sequencing, and 5 are partially methylated 6 mA and 4mC motifs having uncertain identities thus not selected into the list of confident motifs.

A novel method for de novo methylation typing and fine mapping. Although 45 of the 46 known motifs have already been re-discovered de novo in the above analysis, two critical additional features are yet to be defined: methylation type and methylated base within each motif. Although the t-SNE analysis reveals a lack of a common signature for each methylation type and a large variation in current differences across different motifs of the same methylation type, it shows that DNA methylation events of the same type generally cluster well (FIG. 2C). We hypothesized that a classification model trained using diverse methylation types and motifs may serve as a reliable approach for categorizing de novo detected methylation into a specific methylation type.

In standard applications of classification models, both training and test samples need to be defined with respect to a consistent feature vector (e.g. current differences near methylated bases in our case). However, while both methylation type and methylation position are known for well-characterized training samples (i.e. feature vectors can be consistently defined for classifier training), test samples are not readily aligned consistently because the methylated position is yet to be discovered to mimic practical application for de novo methylation discovery. Essentially, methylation type classification and methylation fine mapping are coupled problems that need to be approached simultaneously.

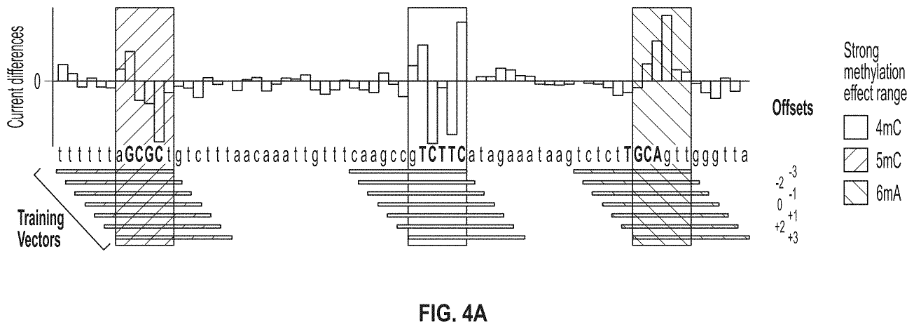

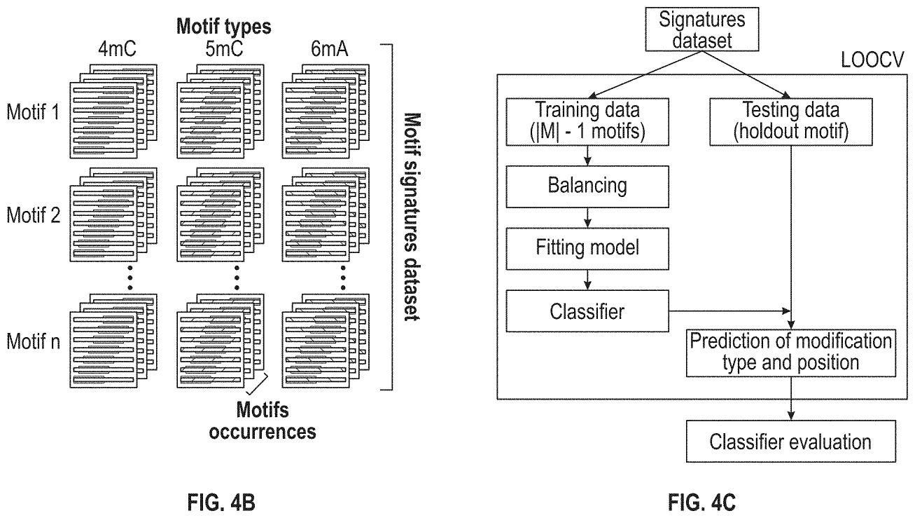

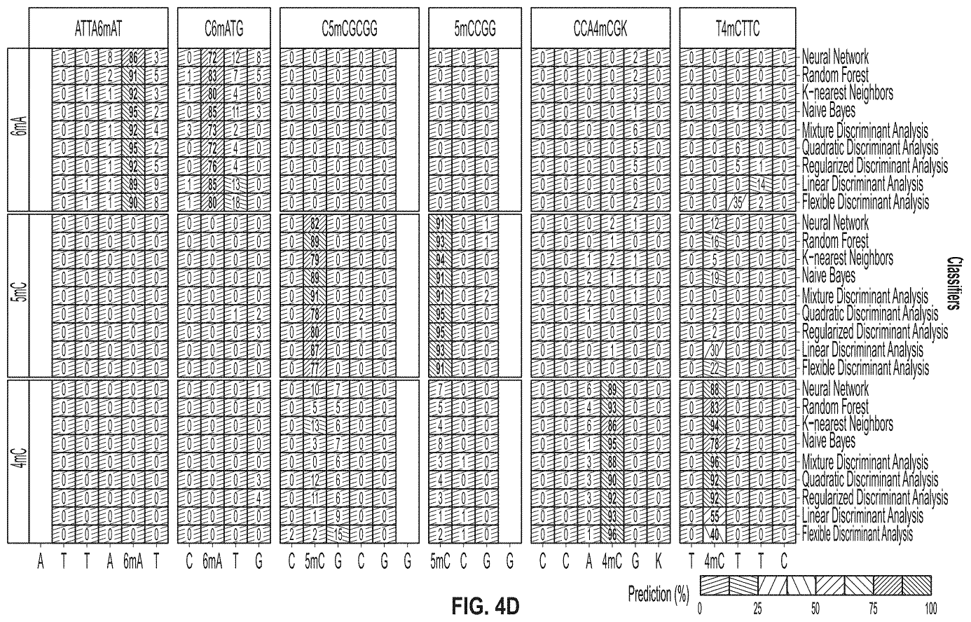

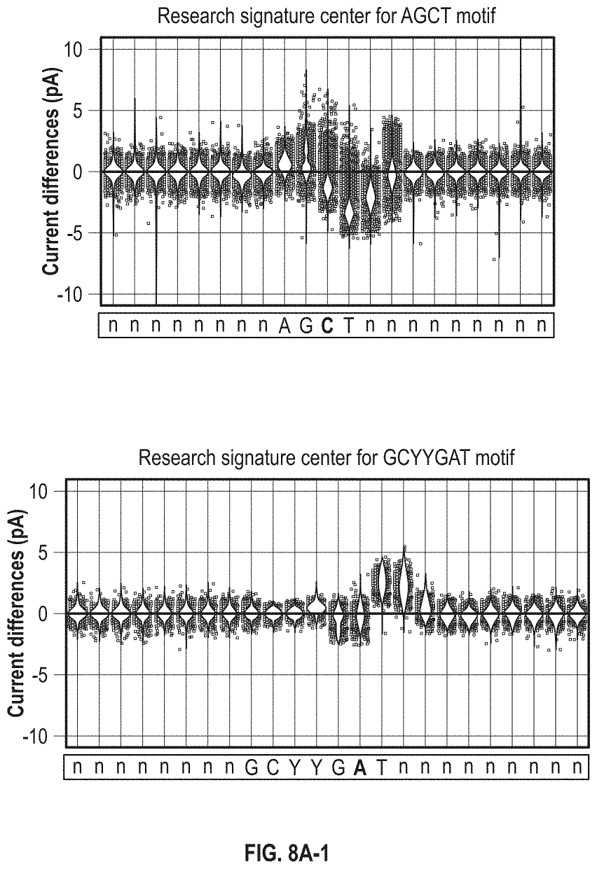

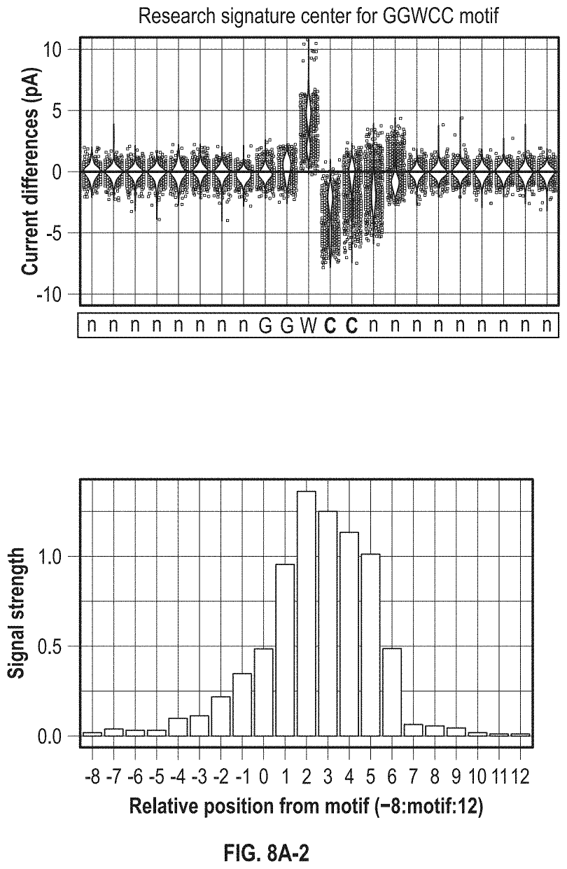

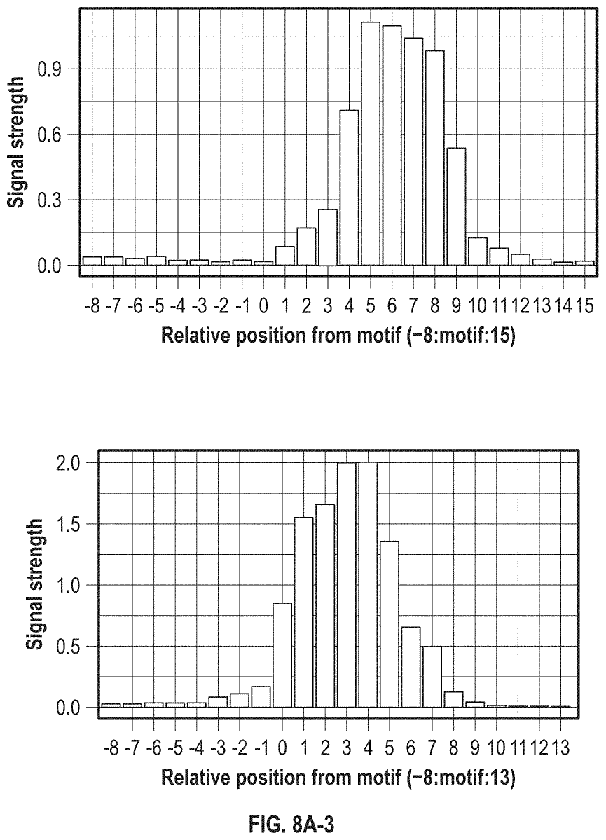

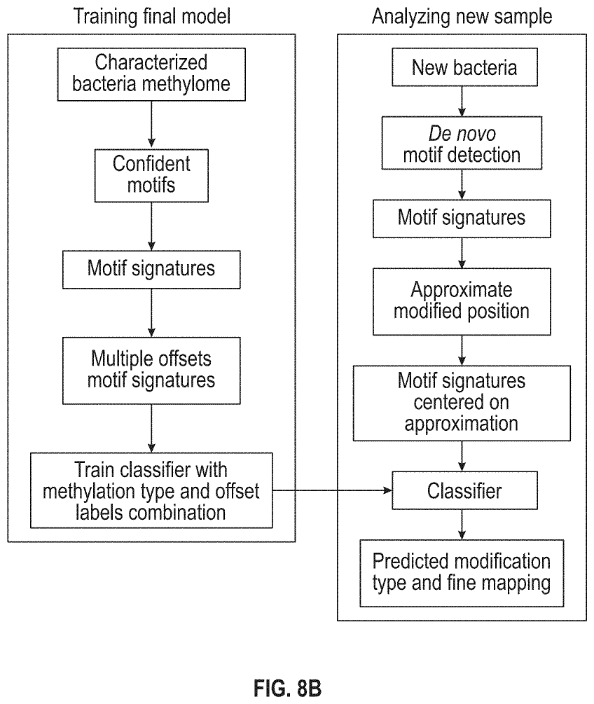

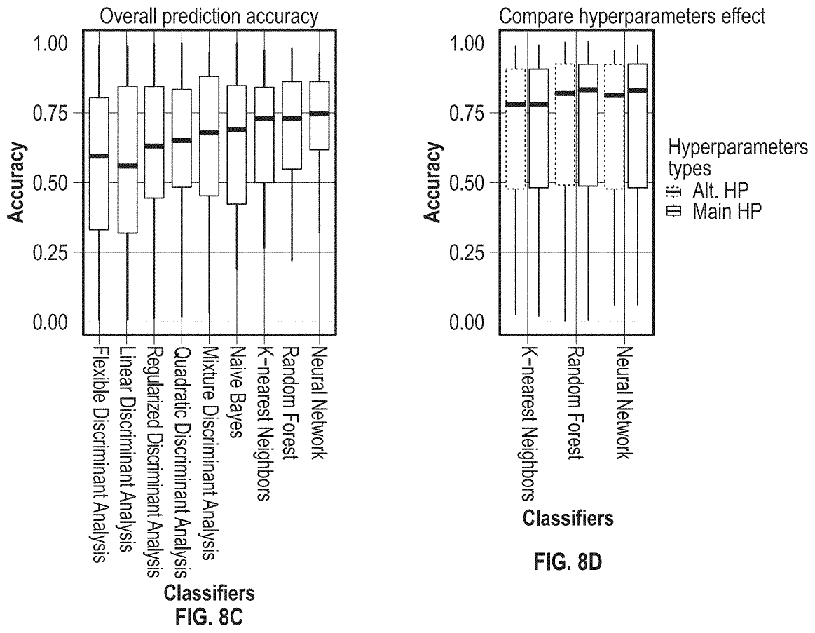

Encouragingly, although the methylated base is not always at the center of the current differences, we did observe a relatively narrow window of no more than +/−3 bp offsets from peak centers across the 45 well-characterized motifs (FIG. 8A). This motivated us to design a novel classifier training strategy in which each well-characterized methylation occurrence is represented by multiple feature vectors with offsets relative to the known methylation position (+/−3 bp). Each methylation occurrence from a wide range of sequence context is learned 7 times by the classifier, each time using current differences at a specific offset from the methylated base. For a given test sample with unknown methylation type and unknown methylated position, the classifier will first take the center of current differences as an approximation of the methylated position and then predict the methylation type and the exact methylated position (FIGS. 4A-4C). This is the core design that enables completely de novo methylation typing and fine mapping, which is critical for practical applications to unknown bacterial genomes.

A set of nine different classifiers was separately trained using current differences flanking known methylated bases following the offset strategy described above (FIGS. 4A-4C; FIG. 8B). For classifier evaluation, we used leave-one-out cross validation (LOOCV) strategy where one motif is held out for testing while all the other 44 motifs are used for training. LOOCV strategy is a good way to show how classifier will behave when used for de novo methylation typing and fine mapping. Considering the different abundance of the three types of DNA methylation, training datasets are balanced across methylation types to avoid the bias of skewed labels in classifier training and testing. With all held out individual methylation sites belonging to a single methylation motif classified, predicted methylated type and position within motif was determined by using the consensus across tested occurrences (Methods). Overall results are largely consistent across the nine classifiers both in terms of accuracy for classifying individual methylation sites (FIG. 4D) and methylation motifs, although k-nearest neighbors, random forest, and neural network had relatively better performances with 95.5% of motifs correctly typed and fine mapped (FIGS. 8C and 8D).

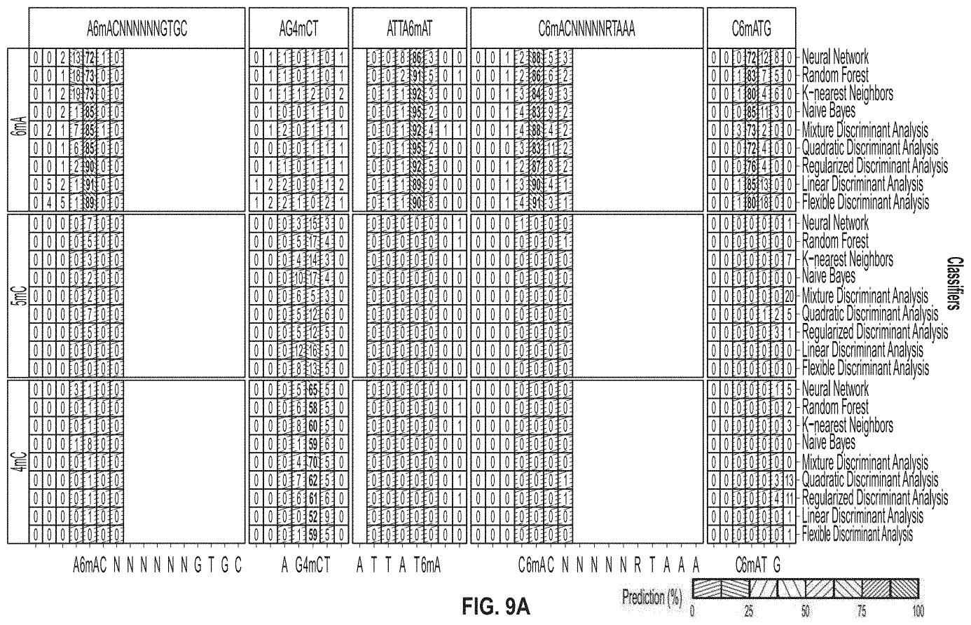

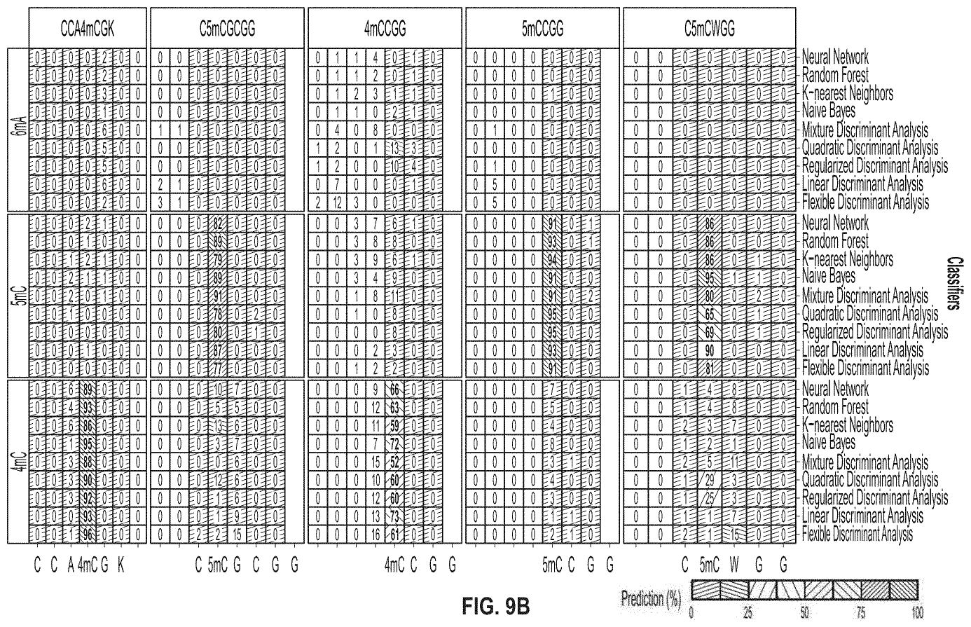

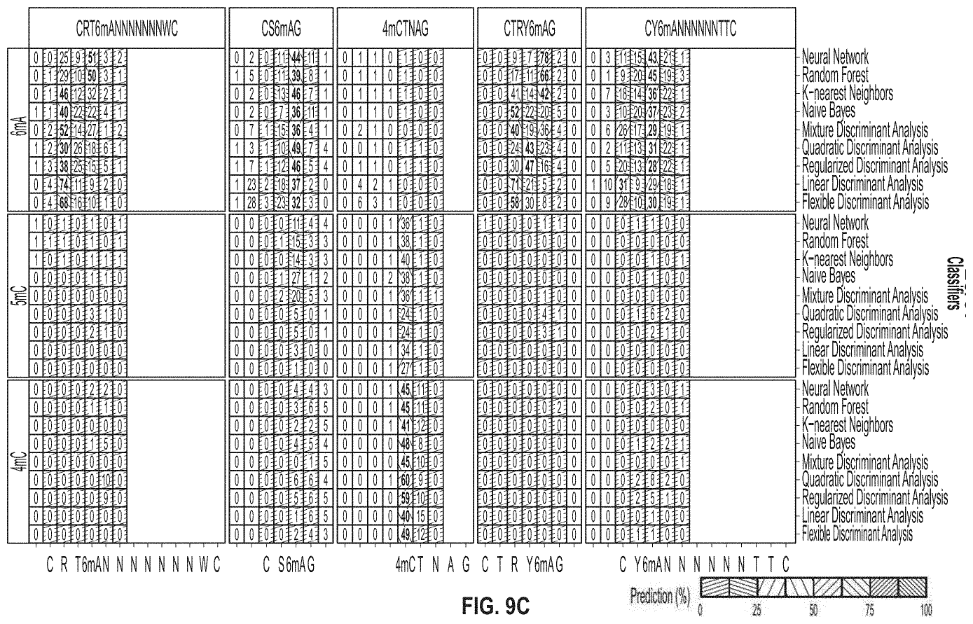

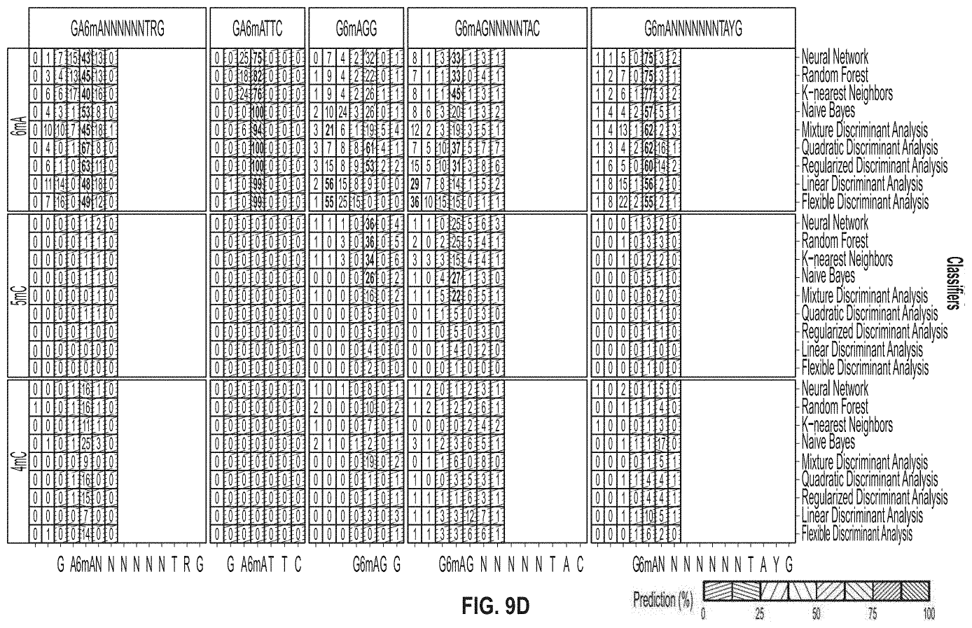

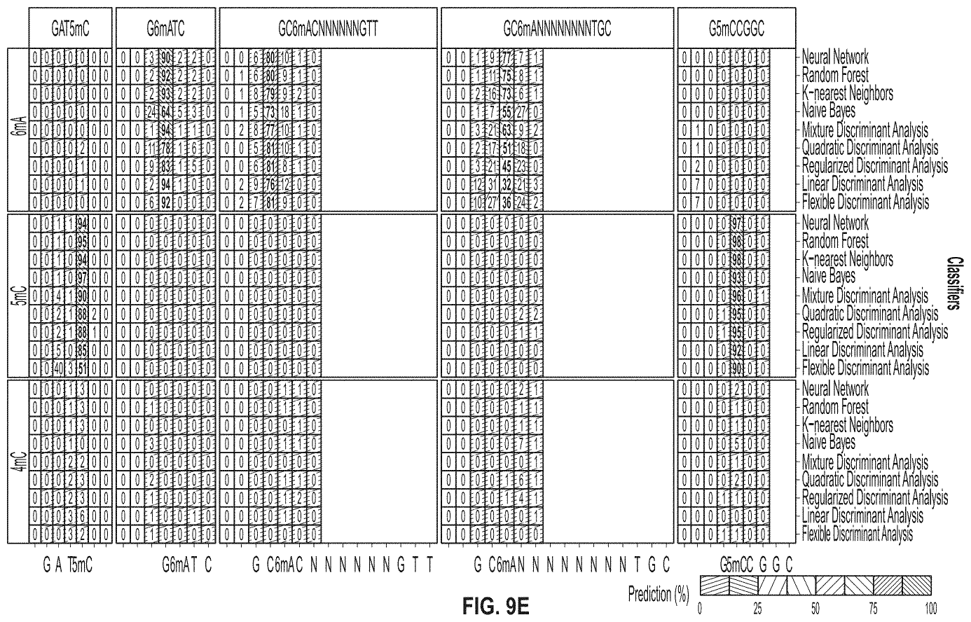

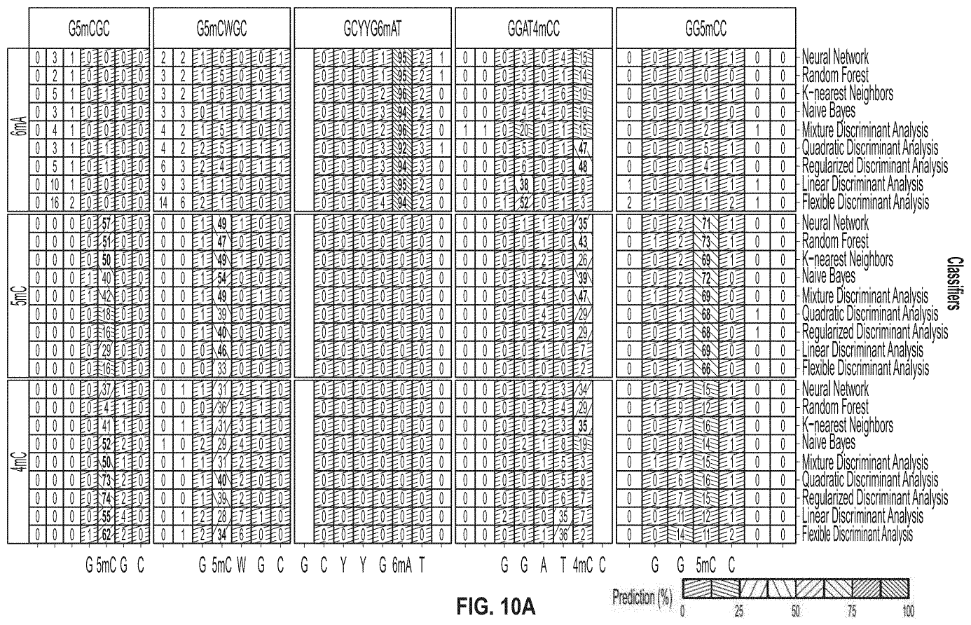

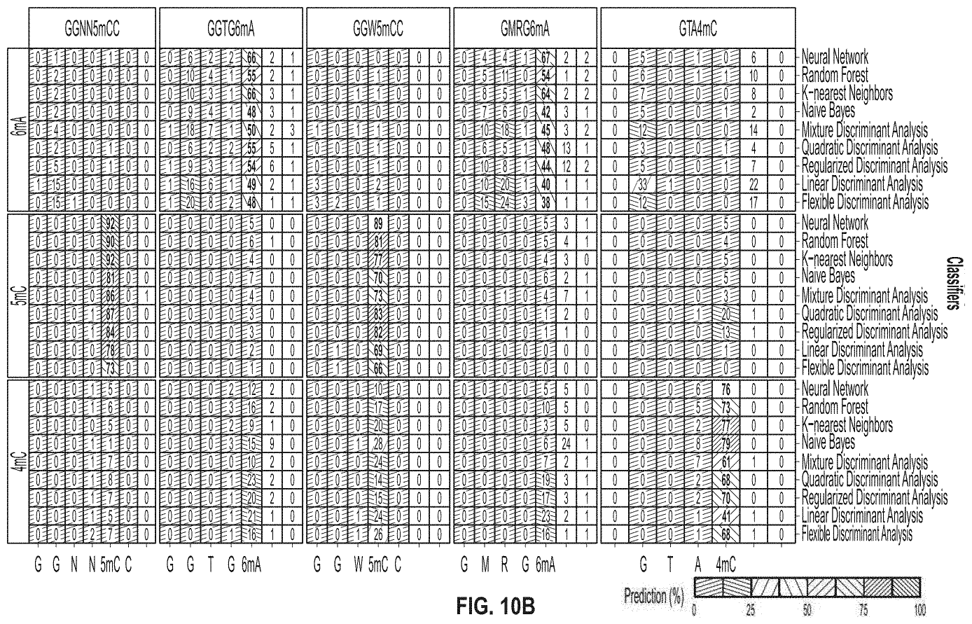

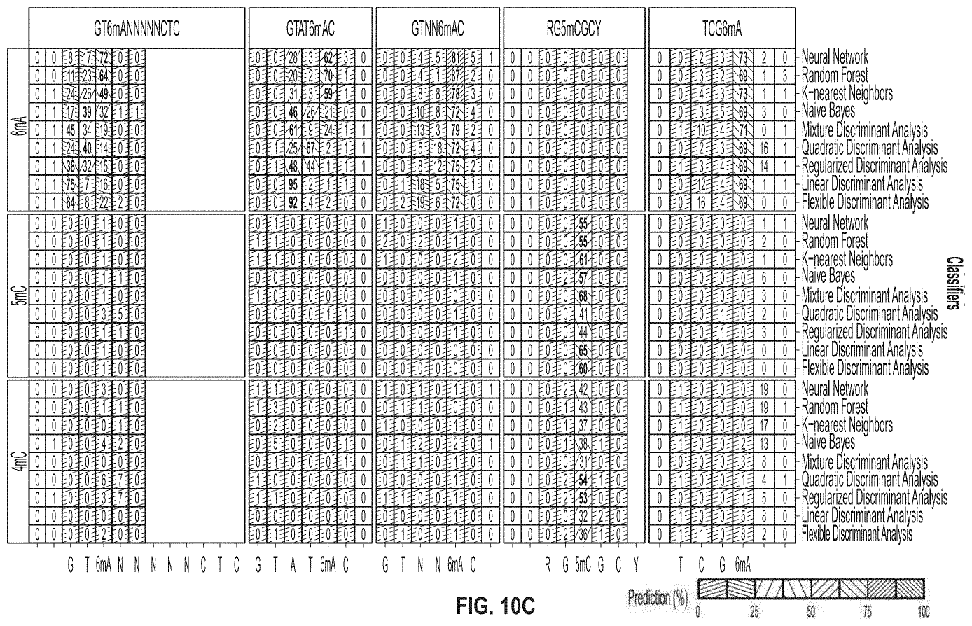

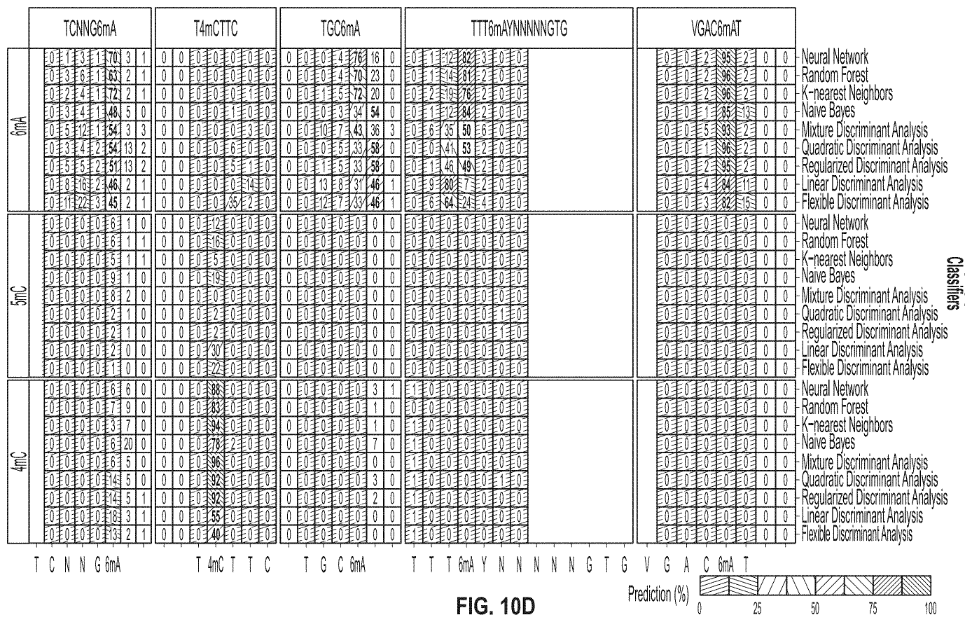

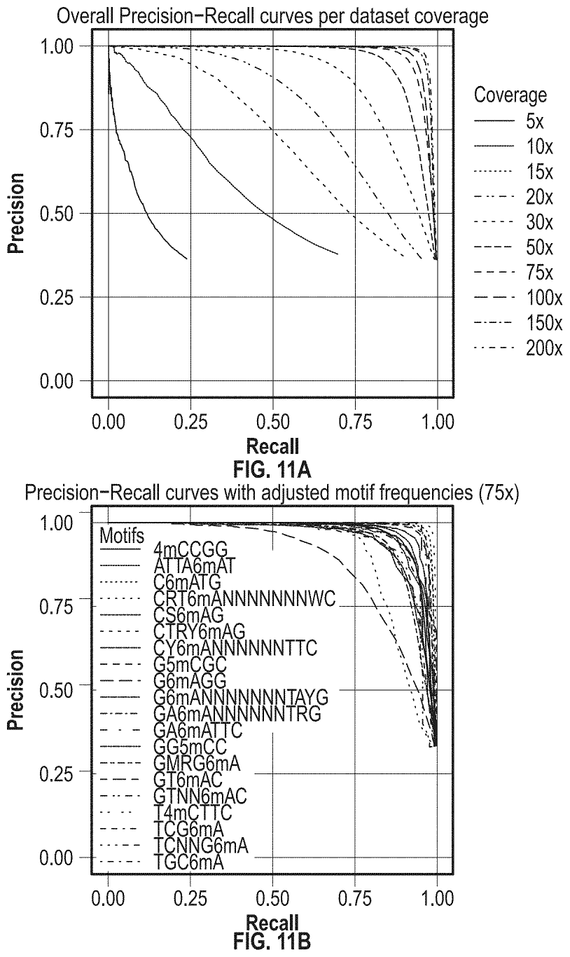

In summary, we developed a new classification-based method that not only captures the complex variation of current differences across methylation types and motifs, but is also trained using a design that allow fine mapping of the methylated base in methylation motif. While we expect the method is highly reliable for de novo methylation typing and fine mapping for a methylation motif (95.5% accuracy), we would like to note that the accuracy for individual methylation event varies dramatically across different motifs, ranging from 26% for G6mAGG to 98% for G5mCCGGC (FIG. 9 and FIG. 10), which is consistent with the observation that motifs of the same methylation type can have different signatures (FIG. 2C; FIGS. 11A and 11B).

### Example 4. De novo methylation motif detection with MEME

Running time for motif discovery with MEME increases with the number of input sequences therefore we limited the number of input sequences used to 2000 with the current implementation and parameters used. Furthermore, we observed that, with some genomes, top peaks could be enriched in specific motifs combination (i.e. motifs in close proximity) preventing MEME from discovering individual motifs in favor of the specific motifs combination. This is due to larger than average smoothed p-value happening when two motif occurrences are near each other, which affect current in a broader genomic region. This phenomenon was observed for genomes with multiple frequent motifs. To limit this bias when observed, we provide an option to randomly select sequences among peaks above a threshold resulting in more than 2000 peaks, effectively avoiding the enrichment of specific motif combination.

Additional information for methylation motif validation. Our de novo methylation motif detection analysis also discovered six motifs absent from our confident list. Two motifs were discovered in H. pylori (i.e. GGWTAA and GGWCNA, likely 6 mA on sixth position) but the analysis of SMRT sequencing data suggest that they are partially methylated. Two additional motifs were found in N. gonorrhoeae. One of them is GTANNNNNCCC, likely modified by the MTase of GT6mANNNNNCTC, but SMRT data show that it's also partially methylated. The other one is TCACC, a 5mC methylation motif according to our classification (i.e. T5mCACC), which would explains why it was not detected with SMRT sequencing analysis. Finally, YGGCCR and WGGCCW were discovered in B. fusiformis and C. perfringens respectively. While both were expected to be the non-degenerated methylation motifs GG4mCC, SMRT sequencing data analysis also suggests that they were also partially methylated explaining our results.

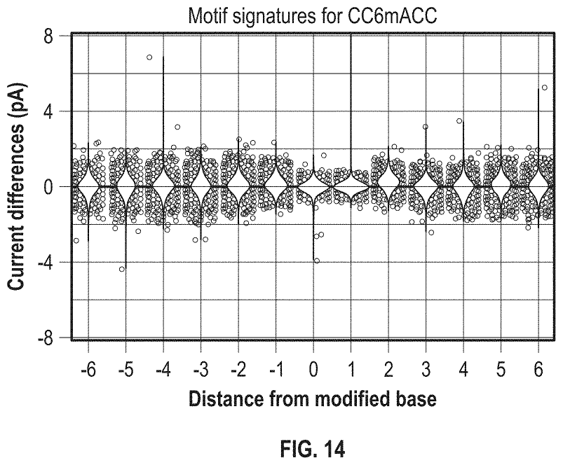

Other unconfident methylation motifs were found only with SMRT sequencing. In H. pylori, we listed three unconfident motifs (i.e. CTGG6mAG, CCTCT6mAG, and STA6mATTC) with weak signals suggesting that they were false discovery or at least partially methylated motifs, thus not suitable for our study. However, we also found a methylation motif in N. gonorrhoeae with strong SMRT sequencing signal (i.e. CC6mACC) while little to no sign of methylation are visible with ONT analysis (i.e. no perturbation in average current differences near motif; FIG. 14). It's unclear if this particular methylation motif is not detected because ONT method is not sensitive to change in nucleotide (between A and 6 mA) in CCACC sequence context or because it's not methylated in our N. gonorrhoeae sample thus it was not used in our analysis.

Note that all motifs mentioned in this section were treated as potential methylation motifs when removing overlapping signal in order to avoid possible compound effects. However, they were ignored from all analysis.

### Example 5. Limiting Factor for Methylation Motif Detection

Genomic coverage strongly affects methylation motif detection ability with substantial improvement in motifs enrichment up to 150× in H. pylori with 20% to 90% of motif detected by increasing coverage from 5× to 150× (FIG. 11A). Overall, 75× (37.5× per strand) is sufficient to detect 100% and 90% of motifs in E. coli and H. pylori respectively. In addition, we observed variation in enrichment across motifs even when variation in motifs frequency was accounted for (FIG. 11B). Motif specific performances depend on the amount of current perturbation introduced by the methylation compared to the non-methylated signal. For example, the G6mAGG motif signature displayed weak current differences and was not detected for H. pylori dataset at lower coverage (<20×). At lower coverage, undetected motifs can display a clear signature although not sufficient to be enriched enough to detect them. Finally, in practice, bacterial methylation motifs have various frequencies in genomes sometimes independent of their complexity, which seems to be a limiting factor for their detection (e.g. GT6mAC in H. pylori). Note that while methylation motif signatures represent how DNA methylation affect ionic current in a specific genomic context during sequencing, some of their characteristics depend on the data processing method used (e.g. base caller, reads mapper, event aligner, and normalization). We expect that methylation motif detection performance will increase with improvement of nanopore sequencing preprocessing methods, notably for base calling and signal alignment to a reference sequence.

### Example 6. Approximation of Methylated Position from Motif Signature

Our current method for approximating methylated position within de novo detected motifs relies on the identification of the center of the motif signature. However, other educated guesses could be made based on motif signature and refining plots, which would permit reducing the DNA methylation position research space. First, main current differences are in the [−2 bp, +3 bp] range from the methylated base meaning that for bipartite motifs one could ignore part of the motif depending on which specificity subunit is aligned with current differences. Similarly, this could be done for long motifs if current differences are at one of the motif extremities. This phenomenon is indirectly used in our approximation approach. Second, motif signatures display important variation when the methylated base is close to non-fixed bases, i.e. next to a degenerated base or near motif extremities. This strategy was not used in the current implementation.

### Example 7. Mock Microbiome from Individual Bacteria

In order to define motif selection procedure for contig methylation binning, we constructed a mock metagenome assembly from our individual bacteria reference genomes. Reference genomes were fragmented following mouse gut metagenome contig length distribution from previous SMRT study. Nanopore sequencing native and WGA datasets subsampled at a coverage of 50× were then mapped on the mock metagenome assembly and processed similarly to individual genomes to generate current differences and associated U test p-values (Methods). Possible methylation motifs from the initial set (n=210,176) are scored for long contigs (>=500 kbp) according to the procedure described in Methods. Rules for methylation motif features selection were defined to enrich the final list in known methylation motifs from bacteria in the mock community. Only genomic positions with 10× coverage were scored in both scoring steps.

We applied the following cutoff on methylation features: minimum absolute current differences (1.5), minimum number of motif feature occurrences per confident contigs (20), minimum number of significant features in bipartite motifs (2), and discard overlapping motifs (bipartite motif explained by 4 to 6-mers motifs). Any motif features satisfying those requirements are scored in remaining contigs. Mouse gut metagenome binning was processed with same parameters except that motif feature scores from contigs with few occurrences (less than 5) were set at 0 to account for a noisier signal from real microbiome data.

### Example 8. Methylation Discovery from Microbiome and Methylation-Enhanced Metagenomic Analyses

Because uncultured bacteria likely represent a significant proportion of the overall diversity of bacterial DNA methylation, we further attempted to perform de novo methylation discovery and characterization from a mouse gut microbiome using nanopore sequencing. For microbes with fairly high abundance, metagenomic assembly often generates reasonably long contigs, which can be technically treated as individual genomes for methylation analysis using the procedure described in the last section. However, for microbes with relatively lower abundance, metagenomic assembly often results in fragmented genomes where contigs are short hence including only a limited number of occurrences of each motif, which makes methylation motifs discovery statistically underpowered if each metagenomic contig is examined separately.

Fragmentation related issues can be mitigated by using diverse binning methods intended to group related contigs together (species or strains level). Those methods encompass sequence composition features binning, contig coverage binning, as well as chromosome interaction maps.

Recent work demonstrates that microbial DNA methylation can be exploited to enhance the grouping of metagenome contigs (i.e. methylation binning) using SMRT sequencing. Instead of trying to discover precise methylation motifs from individual contigs, the methylation binning method presented in this recent work computes 6 mA profiles (methylation scores for putative 6 mA motifs) for each contig and then groups contigs together into bins based on methylation profiles similarities. We hypothesized that methylation binning of metagenomic contigs could be done using nanopore sequencing, which holds great promise due to its sensitivity for detecting all three types of common DNA methylations (4mC, 5mC, and 6 mA) beyond the scope of work that focused on 6 mA alone, especially because SMRT sequencing does not effectively detect 5mC.

We first developed a new methylation binning method specifically for nanopore sequencing data considering the fundamental differences from SMRT sequencing. In a nutshell, several important technical steps needed to be developed for nanopore sequencing data because the current differences associated with each of the three types of methylation are spanning multiple events near methylated bases (FIG. 2A, FIG. 3A, and FIGS. 6A-6C) rather than as confined to a single base for 6 mA or 4mC as in SMRT sequencing. After prototyping and evaluation on a mock community, we applied the methylation method to new nanopore sequencing data of the same mouse gut microbiome sample used in the SMRT sequencing-based study. To summarize, we computed methylation feature vectors for a large set of candidate methylation motifs (n=210,176), motifs with informational feature (i.e. significant current differences) were first selected based on large contigs, and methylation feature vectors were then computed in remaining contigs. Methylation feature vectors are then arranged in a methylation profile matrix, which is further used to group contigs with similar methylation profile. To focus on methylation analysis and to ease comparison between nanopore sequencing and SMRT sequencing, we used the SMRT metagenomic assembly reported in the recent study (Methods).

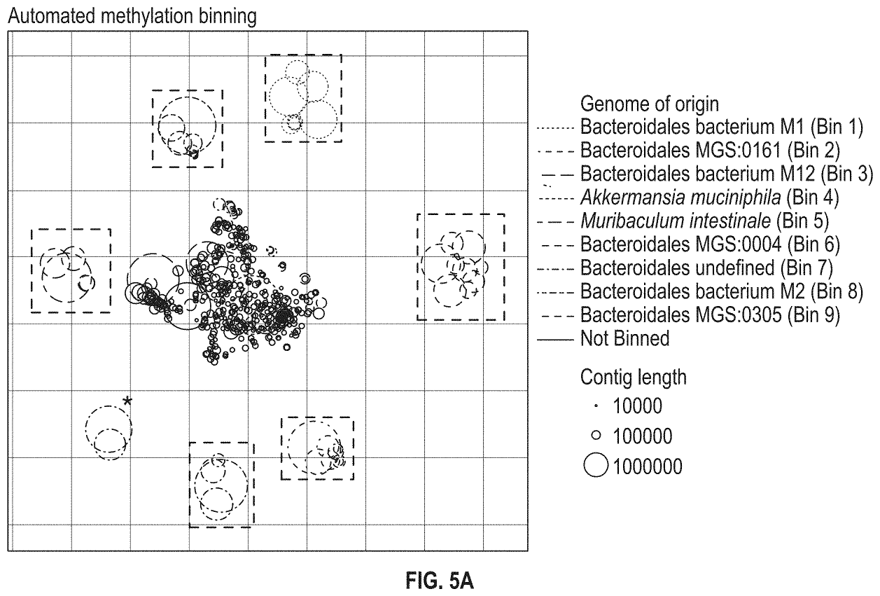

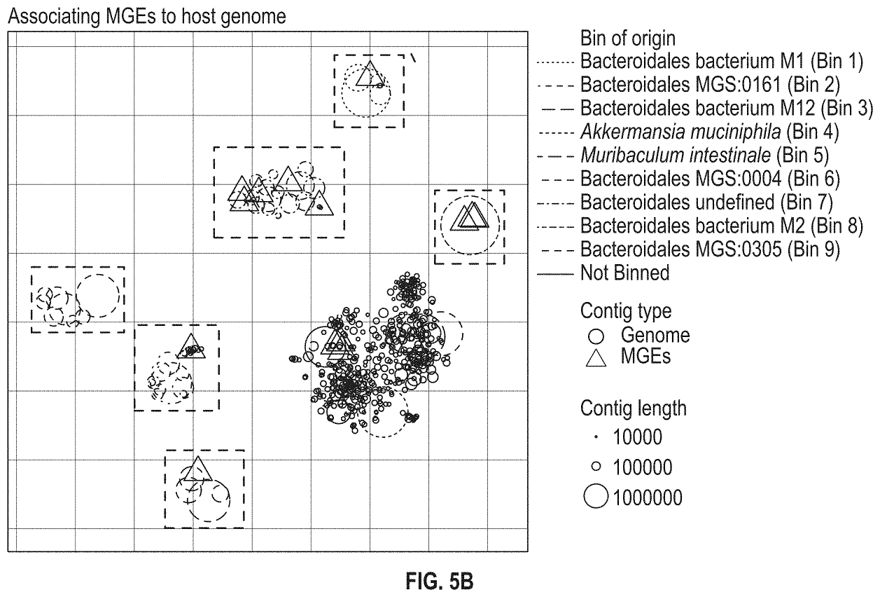

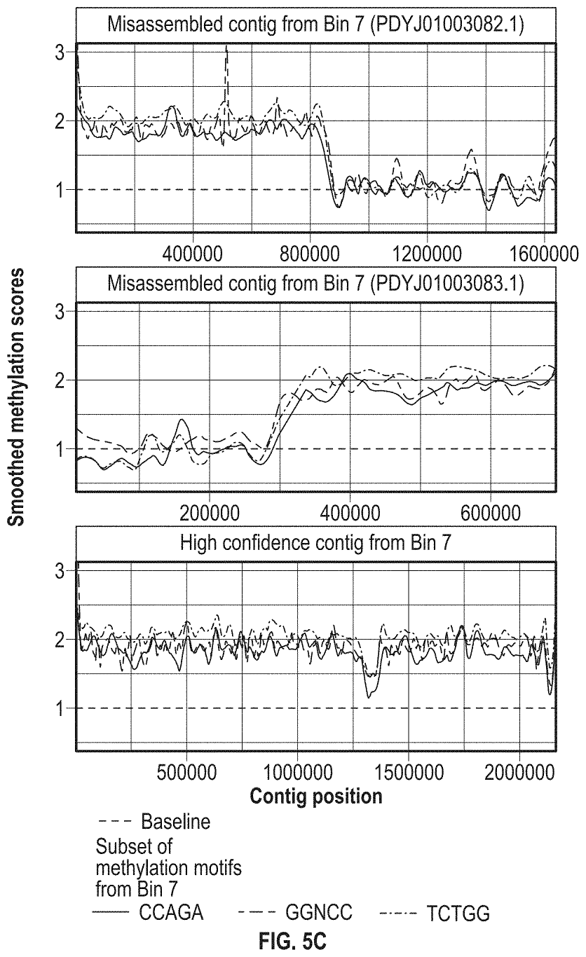

Methylation binning of the mouse gut microbiome sample with nanopore sequencing data revealed seven bins with two to nine contigs in each (FIG. 5A; Table 4).

TABLE 4
Contigs methylation binning results from nanopore sequencing data analysis.
Contigs from metagenome SMRT assembly were used (GCA_002754755.1).
Usage of the contigs for motif detection procedure was also indicated.

| | | Contig | Motif | | | Contig | Motif |
|---|---|---|---|---|---|---|---|
| Bin | Contig | length | detection | Bin | Contig | length | detection |
| Bin 1 | PDYJ01003084. [symbol] | 1128400 | Yes | Bin 6 | PDYJ01001531.1 | 1159367 | Yes |
| | PDYJ01000766. [symbol] | 1089244 | Yes | | PDYJ01002305.1 | 793618 | Yes |
| | PDYJ01000767. [symbol] | 689261 | Yes | | PDYJ01001532.1 | 764722 | Yes |
| | PDYJ01000006. [symbol] | 391145 | Yes | | PDYJ01003086.1 | 410528 | Yes |
| | PDYJ01002309. [symbol] | 231307 | No | | PDYJ01001534.1 | 340141 | Yes |
| | PDYJ01002311. [symbol] | 146864 | No | | PDYJ01001535.1 | 323383 | Yes |
| | PDYJ01000774. [symbol] | 109822 | No | | PDYJ01001537.1 | 189760 | No |
| | PDYJ01000013. [symbol] | 90936 | No | | PDYJ01000772.1 | 173204 | No |
| Bin 2 | PDYJ01001530. [symbol] | 2164130 | Yes | | PDYJ01000773.1 | 120980 | No |
| | PDYJ01002307. [symbol] | 460937 | Yes | Bin 7 | PDYJ01000763.1 | 2165375 | Yes |
| | PDYJ01000770. [symbol] | 323727 | Yes | | PDYJ01000764.1 | 751862 | Yes |
| | PDYJ01000009. [symbol] | 222003 | No | | PDYJ01002304.1 | 399150 | Yes |
| | PDYJ01003091. [symbol] | 144627 | No | | PDYJ01000768.1 | 99577 | No |
| | PDYJ01002314. [symbol] | 92048 | No | Bin 8 | PDYJ01002303.1 | 2565370 | Yes |
| | PDYJ01002313. [symbol] | 45927 | No | | PDYJ01002306.1 | 498769 | Yes |
| | PDYJ01000788. [symbol] | 32935 | No | | PDYJ01000769.1 | 381917 | Yes |
| Bin 5 | PDYJ01000002. [symbol] | 1873721 | Yes | | PDYJ01000771.1 | 215040 | No |
| | PDYJ01000004. [symbol] | 619786 | Yes | | PDYJ01000776.1 | 74732 | No |
| | PDYJ01001533. [symbol] | 391705 | Yes | | PDYJ01000036.1 | 32734 | No |
| | PDYJ01001536. [symbol] | 166965 | No | | PDYJ01003099.1 | 27793 | No |
| | PDYJ01003090. [symbol] | 145865 | No | | PDYJ01001709.1 | 24464 | No |
| | | | | | PDYJ01000040.1 | 23989 | No |

[symbol]

indicates data missing or illegible when filed

Through a bin-level comparison, bins from nanopore sequencing data closely matched those from SMRT sequencing data, and none of the nanopore sequencing bins contained misclassified contigs. Consistent between the two technologies, methylation binning effectively separated the multiple Bacteroidetes species (all bins except Bin 4 and 9) that are usually hard to distinguish from each other due to their highly similar genome sequence composition and abundance.

Based on the above methylation binning analysis, contigs larger than 250 kb from the same bin can be combined to enhance the statistical power of methylation motif detection. Collectively, 40 methylation motifs (36 with unique recognition sequences) were discovered from the seven bins (Table 5).

TABLE 5
Motif detection results from metagenome dataset.

| Motif | | Methylation | | | | | |
|---|---|---|---|---|---|---|---|
| Recog- | Motif | Position | Type | | | | Compatible |
| nition | | Predic- | Predic- | Motif | | SMART | detection |
| Sequence | Length | tion | tion | Prediction | Bin | motif | prediction |
| ACCGAG | 6 | 5 | 5mC | ACCG5mCG | Bin 1 | ACCG6mA | No |
| | | | | | | G | |
| ACGGG | 5 | 2 | 5mC | A5mCGGG | Bin 1 | NA | Yes |
| CCCGT | 5 | 2 | 5mC | C5mCCGT | Bin 1 | NA | Yes |
| KCCGGM | 6 | 3 | 5mC | KC5mCGGM | Bin 1 | NA | Yes |
| WCCGGW | 6 | 2 | 5mC | W5mCCGGW | Bin 1 | NA | Yes |
| AGCTC | 5 | 3 | 5mC | AG5mCTC | Bin 2 | NA | Yes |
| CGWCG | 5 | 4 | 5mC | CGW5mCG | Bin 2 | NA | Yes |
| CTGCAG | 6 | 4 | 6mA | CTG6mAAG | Bin 2 | CTGC6mA | No |
| | | | | | | G | |
| GAGCT | 5 | 4 | 5mC | GAG5mCT | Bin 2 | NA | Yes |
| GATC | 4 | 4 | 5mC | GAT5mC | Bin 2 | NA | Yes |
| GGCC | 4 | 3 | 5mC | GG5mCC | Bin 2 | NA | Yes |
| RCCGGY | 6 | 2 | 5mC | R5mCCGGY | Bin 2 | NA | Yes |
| AGCANN | 13 | 4 | 6mA | AGC6mANN | Bin5 | Yes | Yes |
| NNNNRT | | | | NNNNRT | | | |
| C | | | | C | | | |
| GAYNNN | 13 | 2 | 6mA | G6mAYNNN | Bin 5 | Yes | Yes |
| NNNTGC | | | | NNNTGC | | | |
| T | | | | T | | | |
| AACAGC | 6 | 3 | 6mA | AA6mAAGC | Bin 6 | AAC6mAG | No |
| | | | | | | C | |
| ATGCAT | 6 | 5 | 6mA | ATGC6mAT | Bin 6 | Yes | Yes |
| AYCNNNN | 11 | 1 | 6mA | 6mAYCNNN | Bin 6 | Yes | Yes |
| RTAG | | | | NRTAG | | | |
| CCNGG | 5 | 2 | 5mC | C5mCNGG | Bin 6 | NA | Yes |
| CGWCG | 5 | 4 | 5mC | CGW5mCG | Bin 6 | NA | Yes |
| CTAYNN | 11 | 3 | 6mA | CT6mAYNN | Bin 6 | Yes | Yes |
| NNGRT | | | | NNGRT | | | |
| GAGCCC | 6 | 4 | 5mC | GAG5mCCC | Bin 6 | NA | Yes |
| GAGCTC | 6 | 4 | 5mC | GAG5mCTC | Bin 6 | NA | Yes |
| GATC | 4 | 4 | 5mC | GAT5mC | Bin 6 | NA | Yes |
| GCWGC | 5 | 2 | 5mC | GsmCWGC | Bin 6 | NA | Yes |
| GGGCTC | 6 | 4 | 5mC | GGG5mCTC | Bin 6 | NA | Yes |
| GGNCC | 5 | 4 | 5mC | GGN5mCC | Bin 6 | NA | Yes |
| GGNNCC | 6 | 5 | 5mC | GGNN5mCC | Bin 6 | NA | Yes |
| ACAYNN | 14 | 3 | 6mA | AC6mAYN | Bin 7 | Yes | Yes |
| NNNNNT | | | | NNNNNNT | | | |
| GG | | | | GG | | | |
| CCANNN | 14 | 3 | 6mA | CC6mANN | Bin 7 | Yes | Yes |
| NNNNRT | | | | NNNNNRT | | | |
| GT | | | | GT | | | |
| CCAGA | 5 | 2 | 5mC | C5mCAGA | Bin 7 | NA | Yes |
| GGCAGC | 6 | 3 | 6mA | GG6mAAGC | Bin 7 | Yes | Yes |
| GGNCC | 5 | 4 | 5mC | GGN5mCC | Bin 7 | NA | Yes |
| GTGATG | 6 | 4 | 6mA | GTG6mATG | Bin 7 | Yes | Yes |
| RCCGGY | 6 | 2 | 5mC | R5mCCGGY | Bin 7 | NA | Yes |
| TCTGG | 5 | 2 | 5mC | T5mCTGG | Bin 7 | NA | Yes |
| AGATG | 5 | 3 | 6mA | AG6mATG | Bin8 | Yes | Yes |
| CCCGC | 5 | 2 | 5mC | C5mCCGC | BinS | NA | Yes |
| CCWGA | 5 | 2 | 5mC | C5mCWGA | Bin S | NA | Yes |
| GCGGG | 5 | 2 | 5mC | G5mCGGG | BinS | NA | Yes |
| TCWGG | 5 | 2 | 5mC | T5mCWGG | Bin S | NA | Yes |

Next, we applied the methylation typing and fine mapping method trained in the last section to these 40 methylation motifs and compiled results from k-nearest neighbors, random forest, and neural network. Classifications are consistent with motif recognition sequences and across classifiers for 37 motifs: 10 motifs are identified as 6 mA and 27 as 5mC (Table 5). Absence of 4mC motifs is consistent with the analysis of SMRT sequencing data from the recent study, which also confirmed every 6 mA motif discovered with our method (Methods). The de novo detection of a large number of 5mC motifs is very encouraging because previous large-scale bacterial methylome studies were almost exclusively based on SMRT sequencing, which is known to be ineffective for detecting 5mC methylation.

We further attempted to link mobile genetic elements (MGEs) to their host genome based on their methylation profiles. Using the list of 40 de novo discovered methylation motifs, we found that 11 of the 19 MGEs annotated from this microbiome sample were binned according to their methylation profiles using nanopore sequencing data (five plasmids and six conjugative transposons; FIG. 5B; Table 6), while nine were binned with the SMRT analysis. With eight MGEs binned as with SMRT analysis and three newly binned MGEs, nanopore sequencing increased MGEs linking potential compared to SMRT methylation binning likely owing to its better sensitivity to 5mC motifs.

TABLE 6
Contigs and MGEs methylation binning results from nanopore sequencing data analysis.

| | | Contig | Bin of | Contig | | | Contig | Bin of | Contig |
|---|---|---|---|---|---|---|---|---|---|
| Bin | Contig | length | origin | type | Bin | Contig | length | origin | type |
| Bin 1 | PDYJ01003228.1 | [symbol] | Not binned | genome | Bin 6 | box6_contig_0 | [symbol] | Bin 6 | genome |
| | box1_contig_0 | [symbol] | Bin 1 | genome | | box6_contig_18 | [symbol] | Bin 6 | genome |
| | box1_contig_2 | [symbol] | Bin 1 | genome | | box6_contig_20 | [symbol] | Bin 6 | genome |
| | box1_contig_3 | [symbol] | Bin 1 | genome | | box6_contig_3 | [symbol] | Bin 6 | genome |
| | box1_contig_5 | [symbol] | Bin 1 | genome | | box6_contig_4 | [symbol] | Bin 6 | genome |
| | box1_contig_6 | [symbol] | Bin 1 | MGE | | box6_contig_7 | [symbol] | Bin 6 | genome |
| Bin 2 | PDYJ01000052.1 | [symbol] | Not binned | genome | | box6_contig_8 | [symbol] | Bin 6 | genome |
| | PDYJ01002722.1 | [symbol] | Not binned | genome | | box6_contig_9 | [symbol] | Bin 6 | genome |
| | box2_contig_0 | [symbol] | Bin 2 | genome | Bin 7 | PDY [symbol] | [symbol] | Not binned | genome |
| | box2_contig_10 | [symbol] | Bin 2 | MGE | | PDY [symbol] | [symbol] | Not binned | genome |
| | box2_contig_11 | [symbol] | Bin 2 | genome | | box7_contig_0 | [symbol] | Bin 7 | genome |
| | box2_contig_2 | [symbol] | Bin 2 | genome | | box7_contig_1 | [symbol] | Bin 7 | genome |
| | box2_contig_3 | [symbol] | Bin 2 | genome | | box7_contig_10 | [symbol] | Bin 7 | genome |
| | box2_contig_4 | [symbol] | Bin 2 | genome | | box7_contig_11 | [symbol] | Bin 7 | MGE |
| | box2_contig_5 | [symbol] | Bin 2 | genome | | box7_contig_12 | [symbol] | Bin 7 | genome |
| | box2_contig_6 | [symbol] | Bin 2 | genome | | box7_contig_13 | [symbol] | Bin 7 | genome |
| | box2_contig_8 | [symbol] | Bin 2 | genome | | box7_contig_14 | [symbol] | Bin 7 | genome |
| | box2_contig_9 | [symbol] | Bin 2 | genome | | box7_contig_15 | [symbol] | Bin 7 | MGE |
| | box7_contig_19 | [symbol] | Bin 7 | genome | | box7_contig_16 | [symbol] | Bin 7 | MGE |
| | box7_contig_20 | [symbol] | Bin 7 | genome | | box7_contig_17 | [symbol] | Bin 7 | genome |
| | box7_contig_21 | [symbol] | Bin 7 | genome | | box7_contig_18 | [symbol] | Bin 7 | genome |
| | box7_contig_22 | [symbol] | Bin 7 | genome | | box7_contig_2 | [symbol] | Bin 7 | genome |
| | box7_contig_23 | [symbol] | Bin 7 | genome | | box7_contig_27 | [symbol] | Bin 7 | genome |
| | box7_contig_24 | [symbol] | Bin 7 | genome | | box7_contig_3 | [symbol] | Bin 7 | genome |
| | box7_contig_25 | [symbol] | Bin 7 | genome | | box7_contig_5 | [symbol] | Bin 7 | MGE |
| | box7_contig_26 | [symbol] | Bin 7 | genome | | box7_contig_6 | [symbol] | Bin 7 | genome |
| | box7_contig_28 | [symbol] | Bin 7 | genome | | box7_contig_8 | [symbol] | Bin 7 | genome |
| | box7_contig_29 | [symbol] | Bin 7 | genome | | box7_contig_83 | [symbol] | Bin 7 | genome |
| | box7_contig_30 | [symbol] | Bin 7 | genome | | box7_contig_85 | [symbol] | Bin 7 | genome |
| | box7_contig_31 | [symbol] | Bin 7 | genome | | box7_contig_86 | [symbol] | Bin 7 | genome |
| | box7_contig_35 | [symbol] | Bin 7 | genome | | box7_contig_88 | [symbol] | Bin 7 | genome |
| | box7_contig_38 | [symbol] | Bin 7 | genome | | box7_contig_9 | [symbol] | Bin 7 | MGE |
| | box7_contig_63 | [symbol] | Bin 7 | genome | Bin 8 | box8_contig_0 | [symbol] | Bin 8 | genome |
| | box7_contig_74 | [symbol] | Bin 7 | genome | | box8_contig_1 | [symbol] | Bin 8 | MGE |
| Bin 5 | box1_contig_1 | [symbol] | Bin 1 | MGE | | box8_contig_5 | [symbol] | Bin 8 | MGE |
| | box5_contig_0 | [symbol] | Bin 5 | genome | | box8_contig_5 | [symbol] | Bin 8 | MGE |
| | box5_contig_1 | [symbol] | Bin 5 | genome | | | | | |
| | box5_contig_2 | [symbol] | Bin 5 | genome | | | | | |
| | box5_contig_3 | [symbol] | Bin 5 | genome | | | | | |

[symbol]

indicates data missing or illegible when filed

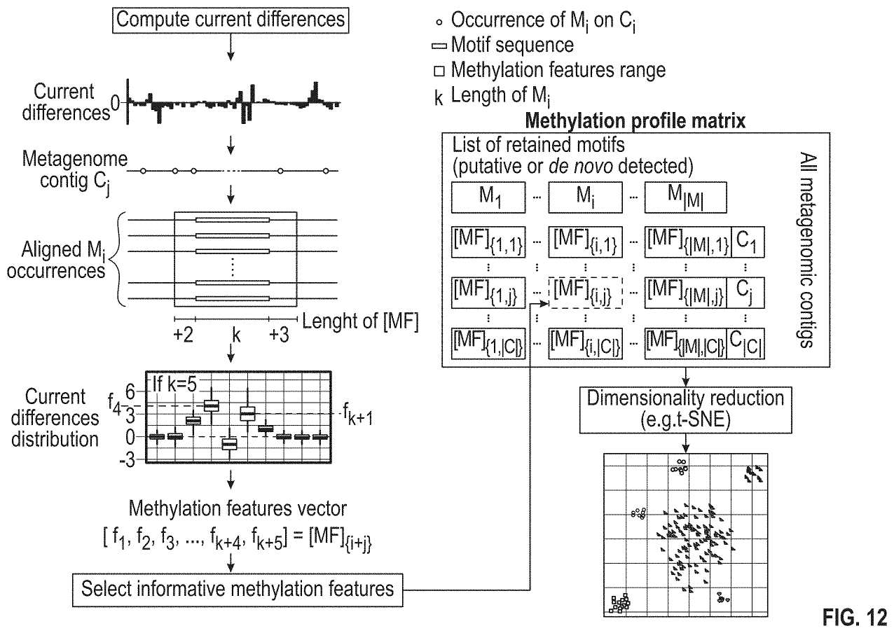

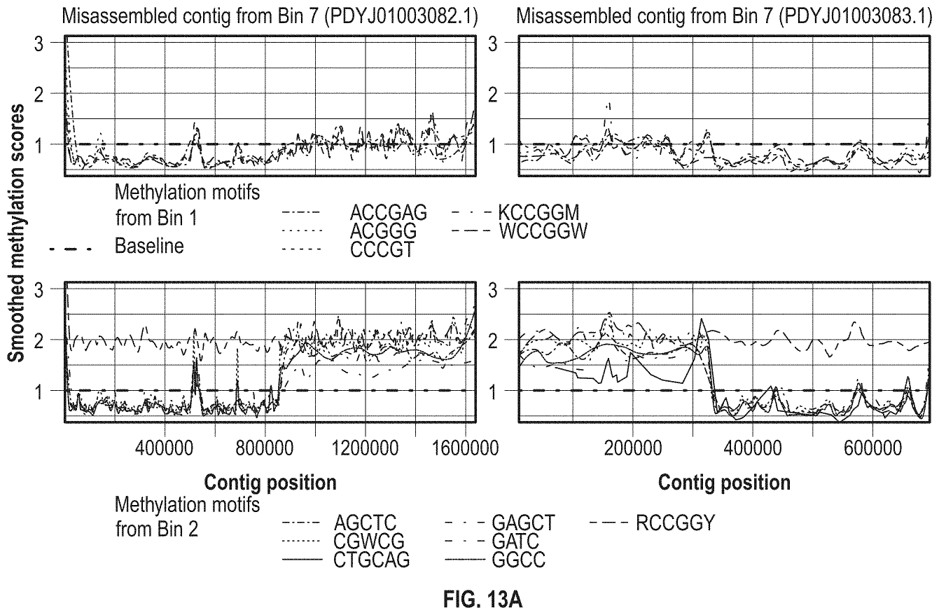

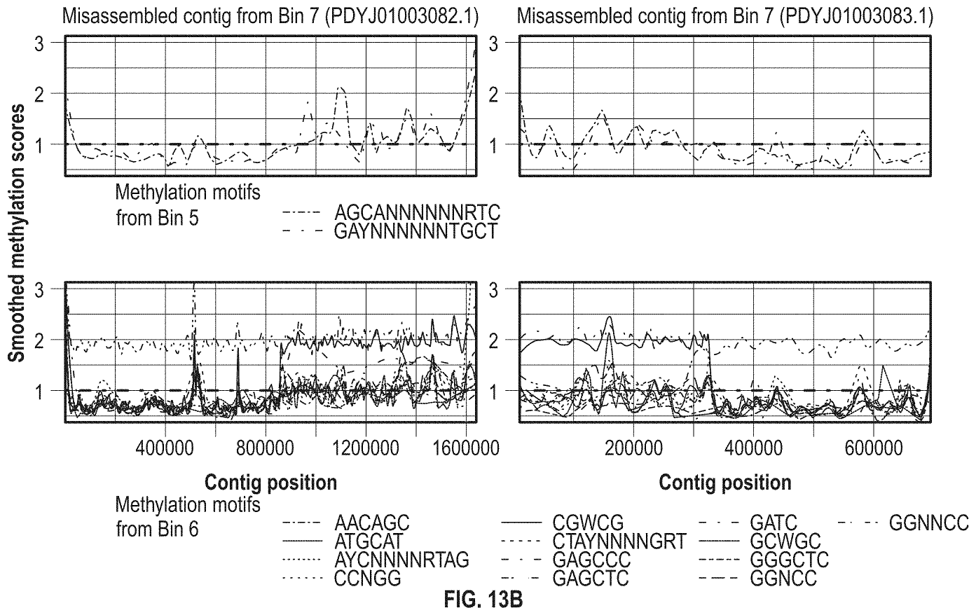

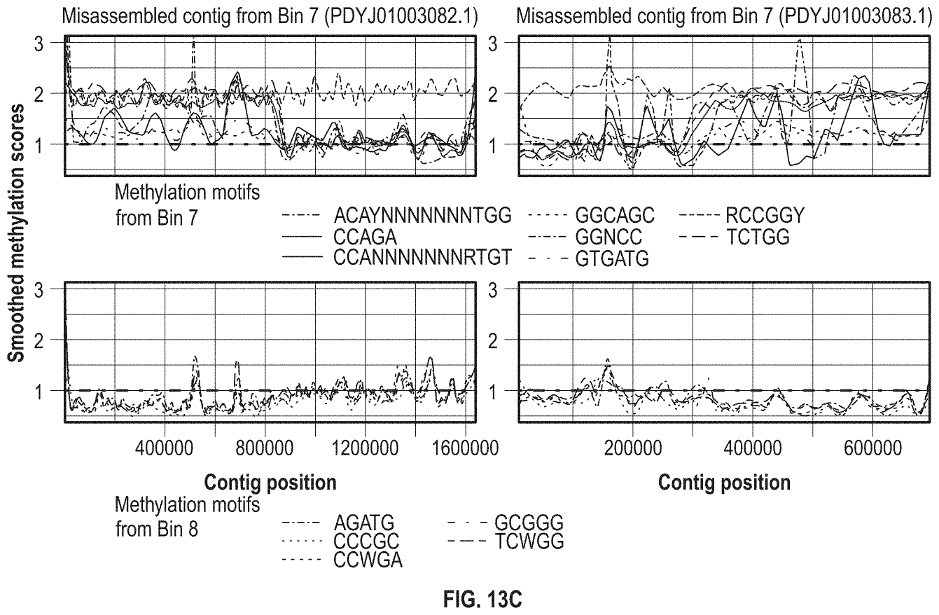

In addition to contig binning, we hypothesized that microbial DNA methylation pattern can also be used to discover misassembled contigs. In a nutshell, methylation pattern is expected to be largely consistent across different regions of an authentic metagenomic contig. Following this rationale, we discovered two contigs (marked by asterisk in FIG. 5A) that both show inconsistent intra-contig methylation status (FIG. 5C). By comparing methylation pattern from methylation motif sets from the other bins, we found that the contigs in question are chimeric contigs representing species of both Bin 7 and Bin 2 (FIG. 12). This is consistent with the previous examination of coverage uniformity and contamination through single-copy gene count, confirming that those contigs annotated as Bin 7 were misassembled by HGAP2 combining parts of Bin 2 and Bin 7 genomes. Generally, this analysis highlights the benefit of incorporating DNA methylation status (ideally all three types: 6 mA, 4mC and 5mC), which not only help better distinguishing microbes species but also help access contigs homogeneity revealing eventual misassemblies, an application particularly useful for the characterization of complex microbiome samples.

### Discussion of Examples

In this work, we developed a novel method for de novo discovery (detection, typing and fine mapping) of three forms of DNA methylation, namely 4mC, 5mC, and 6 mA, and we expect it to be widely used for de novo characterization of unknown bacterial methylomes as increasing number of researchers start to employ nanopore sequencing. Our comprehensive motif profiling and analysis showed that different methylation motifs of the same methylation type could differently impact current levels captured in nanopore sequencing. This observation has important implications for nanopore sequencing based detection of DNA methylation confirming that a rich collection of methylation sequence context is necessary to develop broadly applicable computational methods for methylation discovery, which we achieved through aggregation of a diverse assortment of methylation motifs from bacteria. We performed rigorous method evaluation and demonstrated that the novel method for discovering and exploiting DNA methylation from individual bacteria as well as microbiome.

As we attempted to use the novel method to directly detect DNA methylation and discover methylation motifs from a microbiome, we demonstrated two valuable utilities of DNA methylation analysis by nanopore sequencing for helping to characterize metagenomes. First, we developed a novel method for methylation binning of metagenomic contigs and linking of MGEs to host genomes building on the method reported for SMRT sequencing data and designing multiple technical procedures addressing the unique properties of nanopore sequencing. Second, we demonstrated that examining methylation pattern along assembled metagenomic contigs could help identify chimeric contigs due to metagenomic misassemblies.

While both SMRT sequencing and nanopore sequencing have great promise of direct detection of DNA methylation without the need for chemical conversions, there has not been an in-depth comparison between the two methods. In this aspect, our comparative analysis over the metagenomic contigs binned by methylation motifs detected by the two technologies from the same microbiome sample provided important insights. First, while 5mC is challenging to detect using SMRT sequencing, nanopore sequencing provides reliable 5mC detection, which significantly improved methylation motif discovery from the analysis of the microbiome sample. The large number of 5mC motifs discovered from the mouse gut microbiome sample suggests the prevalence and diversity of 5mC motifs could have been underestimated in the >2,000 bacterial methylome analysis that were almost exclusively based on SMRT sequencing. Second, we found that multiple long and rare methylation motifs well detected by SMRT sequencing in the metagenome analysis were missed by nanopore sequencing, which can be explained by the current differences associated with each of the three types of methylation diffusion to multiple flanking bases in contrast to the fairly high IPD ratios confined to a single methylation site (4mC or 6 mA) for SMRT sequencing. Collectively, these comparisons suggest that SMRT sequencing and nanopore sequencing have their own strengths and limitations; hence the two technologies are expected to complement each other in various applications.

In this work, we focused on bacterial methylomes of individual microbes and microbiome, and we expected the method to be highly reliable for de novo methylation typing and fine mapping for methylation motifs.

Last but not least, although the current study was focused on three types of DNA methylation, the method can be extended for the detection of additional forms of DNA methylation (5hmC, 5fC and 5caC) as well as other forms of DNA chemical modification such as the various forms of DNA damage (including that associated with cancer), and possibly diverse forms of RNA modifications owing to the unique promise of nanopore technology for direct RNA sequencing.

### Methods for Examples

### (a) Software and Data Availability

Software of the novel methods and a tutorial will be made publically available at http://github.com/fanglab/. All sequencing data generated in this study will be deposited in SRA.

### (b) Samples Collection and DNA Extraction

A set of seven bacteria was rationally selected using previous study10 and REBASE20 to provide a large diversity of methylation motifs in particular for the less frequent 4mC and 5mC methylation motifs: Bacillus amyloliquefaciens H, Bacillus fusiformis 122, Clostridium perfringens ATCC 13124, Escherichia coli MG1655 ATCC 47076, Methanospirillum hungatei JF-1, Helicobacter pylori JP26, and Neisseria gonorrhoeae FA 1090.

B. amyloliquefaciens H and B. fusiformis 122 DNA samples were obtained from New England Biolabs (NEB, Ipswich, Mass.). Those for C. perfringens ATCC 13124, M. hungatei JF-1, H. pylori JP26, and N. gonorrhoeae FA 1090 were obtained from the Human Health Therapeutics Research Area at National Research Council Canada, the Department of Microbiology, Immunology, and Molecular Genetics at University of California Los Angeles, the Department of Medicine at New York University Langone Medical Center (NYUMC), and the University of Oklahoma Health Sciences Center, respectively. Finally, we obtained E. coli MG1655 ATCC 47076 directly from the American Type Culture Collection (ATCC, Manassas, Va.).

Mouse gut microbiome DNA sample was obtained from the Department of Medicine at NYUMC and comes from the same mice used in the SMRT sequencing study. Fecal DNA extraction was performed using QIAamp DNA Microbiome Kit (QIAGEN, Hilden, Germany) followed by cleanup with DNA Clean & Concentrator—5 elution buffer (ZYMO Research, Irvine, Calif.) and final elution in 10 mM Tris-HCl, pH 8.5, 0.1 mM EDTA.

### (c) Library Preparation and Sequencing

Quality of input DNA was controlled with Nanodrop 2000 and concentration measured using Qubit 3.0 (Thermo Fisher Scientific, Waltham, Mass.). Native libraries were prepared following 1D Genomic DNA by ligation protocol (SQK-LSK108; version GDE_9002_v108_revT_18Oct2016) with minor modifications described below. Whole genome amplification samples were prepared using REPLI-g Mini Kits (QIAGEN, Hilden, Germany) according to the protocol with 12.5 ng of input DNA and 16 h incubation. Next, WGA samples were treated with T7 endonuclease I (NEB) to maximize nanopore sequencing yield according to ONT documentation. WGA libraries were prepared following Premium whole genome amplification protocol from T7 step (version WAL_9030_v108_revJ_26Jan2017) with minor modifications described below. Bacteria (other than E. coli and H. pylori) and mouse gut microbiome DNA samples, native and WGA, were RNase A treated (FEREN0531, Thermo Fisher Scientific) then fragmented at 8 kbp with g-TUBEs (Covaris, Woburn, Mass.) to homogenized DNA fragments lengths increasing accuracy of input DNA molarity calculation to maximize yields. Final fragment length distributions were determined using Bioanalyzer 2100 (Agilent Technologies, Santa Clara, Calif.). Samples were sequenced on R9.4 and R9.4.1 flow cells.

E. coli and H. pylori libraries (native and WGA) were prepared without fragmentation or Formalin-Fixed, Paraffin-Embedded (FFPE) DNA repair. E. coli and H. pylori WGA input DNA was increased to 3 μg in T7 step with 20 min incubation. Remaining steps were performed according to corresponding ONT protocol and final libraries sequenced on 3 flow cells with a maximum of two consecutive runs per flow cell. Flow cells were washed between runs using the Flow Cell Wash Kit (EXP-WSH002) from ONT. An additional WGA was produced for H. pylori, refer to as independent WGA. Sequencing of native and WGA libraries generated from 289 to 2630× genomic coverage but were down sampled at 200× to more accurately represent common yield targets.

DNA samples for the additional bacteria (B. amyloliquefacien, B. fusiformis, C. perfringens, M. hungatei, and N. gonorrhoeae) were pooled in equimolar quantity for library preparation. Pooling possibility was confirmed by mapping mock ONT reads datasets generated using Nanosim43 (version 1.0.0) on combined references and verifying accurate separation of reads into genome of origin. Native and WGA library preparations were performed using aforementioned ONT protocol and sequenced on two separate flow cells for 48 h each. Sequencing of native and WGA generated datasets with coverage ranging from 102 to 250×.

Finally, mouse gut microbiome libraries were generated according to the One-pot ligation protocol for Oxford Nanopores libraries (dx.doi.org/10.17504/protocols.io.k9acz2e) including the FFPE DNA repair step with exception for the room temperature incubation times that were increased from 10 to 20 minutes. 300 fmol of input DNA were used in FFPE DNA repair steps. Native and WGA libraries were sequenced on two separate flow cells for 48 h each generating 5.0 and 3.1 Gbase of reads respectively with lengths averaging 1.8 and 2.7 kb according to base calling summaries.

### (d) Nanopore Sequencing Signal Processing

Nanopore sequencing reads are base called using ONT Albacore Sequencing Pipeline Software (version 1.1.0). Reads are mapped to corresponding references using BWA-MEM (version 0.7.15 with −x ont2d option). Following steps are performed using R (version 3.3.1)45. Reads are separated by strand according to the initial alignment (package Rsamtools; version 1.24.0)46, and both groups are processed as forward strand reads by mapping reverse strand reads on the reverse complement of the reference genome using BWA-MEM. Supplementary and reverse strand alignments are then filtered out with samtools (version 1.3; flags 2048 and 16)47. Next, events are associated to genomic positions according to alignment coordinates from reads and expected current levels with Nanopolish eventalign (version 0.6.1)14. Event levels are normalized across reads by correcting signal scaling and shifting. Both normalization factors are computed for each read by fitting events level to ONT 6-mer model (nanopolish configuration file r9.4_450bps.nucleotide.6mer.template.model) using robust regression (rlm function). Event level outliers are removed using Tukey's fences methods based on interquartile range (IQR=1.5) for each genomic position. Finally, mean event current differences (pA) were computed by comparing event levels between native sample (maintained methylation state) and WGA sample (essentially methylation free) at each genomic position for both strands separately. This metric is simply referred to as current differences in our manuscript. Associated p-values from two-sided Mann-Whitney U test are also computed (wilcox.test function) which was proposed in Stoiber et al. Only genomic positions with sufficient coverage are considered in later analysis (min_cov=5).

### (e) Motif Enrichment Analysis

DNA methylation affects nanopore sequencing signal at multiple positions around the methylated base (FIG. 2A and FIGS. 6A-6C) meaning detection of methylated sites can be reinforced by combining information from consecutive genomic positions. Consecutive p-values are combined with Fisher's method (sumlog function) in sliding windows (5 bp) smoothing statistical signal along the genome. It combines the methylation related signal near methylated bases and reduces signal noises from spurious genomic positions. Resulting smoothed statistical signals form peaks near methylated positions. Detected peaks are ranked according to their smoothed p-value and those above a chosen threshold are then selected for motif discovery. Corresponding genomic sequences are then extracted (22 bp) and used as input for de novo motifs discovery with MEME software (version 4.11.4; parameters: -dna -mod zoops -nmotifs 5 -minw 4 -maxw 14 -maxsize 1000000). Selection of region of interest based on combined p-values followed by motif detection using MEME was initially proposed in a preprint by Stoiber et al. However, we enhanced the motif discovery potential by closely integrating MEME in our pipeline as described in next paragraphs.

Running time for motif discovery with MEME rapidly increases with size of the sequence dataset to such extend that we had to limit the number of input sequences used. To address this constraint, we adopt a repeated procedure of back and forth between peak detection and motif discovery steps. For each pass, a limited number of input sequences are analyzed with MEME and motifs achieving a sufficient confidence (E-value<=10-30) are reported. After each motif discovery step, peaks explained by discovered motifs, whose corresponding genomic sequence contains at least one of the de novo detected motifs, are removed making it possible to discover less frequent motifs and ones with weaker signals. This repeated procedure is adapted for detecting any number of methylated motifs while decreasing processing time.

Raw motifs called by MEME were further refine by leveraging current difference information. For each motif reported by MEME, we generate a list of mutated motifs by introducing a substitution (one substitution at a time; analysis of GATC will give 12 mutated motifs: AATC, CATC, TATC, GCTC, GGTC, GTTC, GAAC, GACC, GAGC, GATA, GATG, GATT). We then computed each mutated motif signature (see Motifs classification and fine mapping) with associated scores representing total divergence from non-methylated signature (sum of absolute average current differences).

### (f) Parameter Tuning for Signal Processing and Motif Detection

To assess our method performance for de novo motif discovery and tune parameters, we evaluated the enrichment of MEME input sequences for expected motifs as the chosen smoothed p-value threshold varies. Method development and choice of default parameter was guided by evaluating various metrics including Precision-Recall (PR), Receiver Operating Characteristic (ROC) curves and area under curves (AUC). We used the following two comparisons to define contingency table classes: native versus WGA, and independent WGA versus WGA. True positives (TP) and false negatives (FN) are respectively defined as motif occurrences with or without signal peak above threshold in native versus WGA. False positives (FP) are genomic regions without motifs and with signal peak above threshold in native versus WGA as well as motif occurrences with signal peak above threshold in independent WGA versus WGA. Finally, true negatives (TN) are defined as genomic regions without motifs and without peak above threshold in native versus WGA as well as motif occurrences without peak above threshold in independent WGA versus WGA. State of motif occurrences were defined whether a peak was detected above the chosen threshold in a 22 bp window encompassing expected methylated base of motif occurrences. For genomic regions devoid of motif, those were split in 22 bp consecutive units, and used as FP and TN with similar status definition. Performances were computed on first 500 kbp only. When comparing performances for de novo detection between individual motifs, we took into consideration variation in frequencies (i.e. a rare motif will be more difficult to detect). Therefore, in order to make the evaluation more generally applicable, we fixed the ratio of positive regions (22 bp windows from motif occurrences in native versus WGA) over all queried regions to one third by random subsampling, effectively avoiding variation in frequencies across the set of H. pylori motifs.

Using the aforementioned method, we evaluated parameter performances for de novo methylation detection for the following steps or parameters: read mapping, event current normalization, outlier removal, statistical test, p-value combining function, smoothing window size, and peaks window size. We also evaluated the impact of coverage by subsampling at 10 depths ranging from 5× to 200× as well as the impact of motif frequency and the motif specific context (i.e. how methylation type and sequence context affect detection potential; FIGS. 11A and 11B).

### (g) Validation of Methylation Motifs Used for Classification

E. coli and H. pylori were sequenced with SMRT sequencing in order to confirm 4mC and 6 mA methylation motifs using the RS_Modification_and_Motif_Analysis protocol from SMRT Analysis Server (v2.3.0). Methylation status summaries for the remaining bacterial species (modifications.csv and motif_summary.csv files) were obtained from NEB. We confirmed effective methylation of 4mC and 6 mA motifs individually by checking if IPD ratio consistently peaked on expected methylated bases. Finally, REBASE annotation was used as a gold standard for 5mC motifs. Methylation motifs with ambiguous status (e.g. weak or partial IPD ratio peaks) or not reported in REBASE annotation were not used for classifier training.

### (h) Motifs Classification and Fine Mapping

For each bacterial genome, we list methylated genomic positions from each strand based on motif recognition sequences. Methylated positions in close proximity are discarded to avoid introducing unwanted complexity (at least 22 bp apart, each strand considered independently as current signal is strand specific). Ambiguous motifs are removed from any downstream analysis. We extract current differences in [−10 bp, +11 bp] range relative to methylated base positions. Each occurrence is labeled with genome of origin, recognition sequence, methylation type, methylation position within motif, and genomic coordinates. This dataset constitute our methylation motif signatures. Note that for de novo detected methylation motif and refinement function, signatures are generated considering every position in the motif as potentially methylated, which produced a longer signature not necessarily centered on the methylated base.

The training dataset for classification is generated from methylation motif signatures to permit labeling of methylation type and position within motifs simultaneously (FIG. 4A). For each vector of current differences from a methylated site, we generate 7 smaller vectors, lengths 12, offseted by one position so that each of them still contains the [−2 bp, +3 bp] range relative to the methylated base. In other words, those 7 vectors contain current differences from the [−2 bp, +3 bp] range with up to 3 additional position(s) before or after (i.e. [−5 bp, +6 bp]+/−0 to 3 bp). Each of those vectors is labeled with the type of DNA methylation from corresponding motifs as well as corresponding offset used (from −3 to +3) resulting in 21 different labels (7 offsets×3 types DNA methylation).

For the testing datasets, methylated base position is unknown and current difference vectors cannot be defined in the same way. However, methylated base position can be approximate by computing the center of current differences from a motif signature. For that, we average absolute current differences from a motif signature using a sliding window of length 5 and the position with the largest variation is used as an approximation of methylation position within the motif (FIG. 8A). In practice, approximations are not further than 3 bp from the methylated position meaning that the vectors of current differences centered on those approximations will match one type of vector offset used for training because they are generated with −3 to +3 bp offsets.

Prior to any model fitting, the training dataset is balanced, by random sampling, to contain similar number of vectors for each label in order to avoid bias toward the more common methylation type. Classifier hyperparameters (Table 7) were tuned on the balanced training dataset containing all motifs using repeated 10-fold cross validation (n=3) with balanced accuracy (mean and standard deviation) as the main metric.

TABLE 7
Information about classifiers used.

| Model | R Package | R Function | Hyperparameters | Values |
|---|---|---|---|---|
| Neural Network | nnet | nnet | size, decay, maxit | 250, 0.00001, 250 |
| Random Forest | randomForest | randomForest | mtry, ntree | 4, 500 |
| k-Nearest Neighbor Classification | caret | knn3 | k | 10 |
| Native Bayes | klaR | NaiveBayes | usekernel, fL, adjust | TRUE, 0, 1.55 |
| Mixture Discriminant Analysis | mda | mda | nb_subclass | 8 |
| Quadratic Discriminant Analysis | MASS | qda | NA | NA |
| Regularized Discriminant Analysis | klaR | rda | gamma, lambda | 0.03, 0.1 |
| Linear Discriminant Analysis | MASS | lda | NA | NA |
| Flexible Discriminant Analysis | mda, earth | fda | nprune, degree | 21, 1 |

Robustness of chosen hyperparameters was confirmed by comparing performances from three classifiers (k-nearest neighbors, random forest, and neural network) when using parameters either tuned on a dataset containing all motifs (as described above) or a dataset only containing H. pylori motifs only. Both sets of hyperparameters gave similar results when tested on a dataset without H. pylori motifs (FIG. 8D).

Classifier performance evaluation was performed using leave-one-out cross validation strategy (LOOCV) by holding out current differences vectors from one motif and training on remaining vectors (from all motifs except one). The resulting model is then used to predict the label of held out vectors from the tested motif. The LOOCV strategy simulates models behavior when faced with an unseen motif signature. For testing, we only used the set of vectors corresponding to the approximated methylation position found as described previously. Predicted methylated base type for a motif is defined using consensus across all tested motif occurrences. As for methylated base position, the classifier prognosticates the offset between the approximated methylation position chosen as input and the predicted methylation position, which is then converted into position within tested motifs.

### (i) Metagenome Methylation Binning

While methylation motif detection could be performed as for individual bacteria, metagenome assemblies often result in many contigs from multiple organisms with various lengths making individual contig analysis lacking power. Instead, we propose to first bin contigs with similar methylation profiles then perform the motif detection. Nanopore sequencing native and WGA datasets are processed in the same way than for individual bacteria generating current differences alongside metagenome contigs using the existing SMRT metagenome assembly reference (GCA_002754755.1).

For a candidate motif, an associated methylation feature vector is computed by averaging current differences from aggregated occurrences on a metagenomic contig (FIG. 12). Unlike well-characterized methylation motifs, the methylated position in a candidate motif is unknown. Therefore, we consider every position in motifs as potentially methylated by including all potentially affected current differences in the methylation feature vector calculation. For a motif of length k, we compute a methylation feature vector of length k+(2+3), which corresponds to the length of current differences that are possibly affected by a methylated base in a k-mer motif (the core current differences is defined as [−2 bp, +3 bp] range flanking a methylated base). This procedure results in a methylation feature vector of average current differences of length k+5 representing a motif methylation status for a contig. This step represents a major difference from SMRT sequencing based methylation binning method where a single methylation score is generated for a motif on a contig.

The next step is to create a methylation profile matrix comprising methylation feature vectors for each motif of interest in each metagenomic contig, which will be used for methylation binning (FIG. 12). A set of 210,176 candidate motifs is generated according to common structures (4-, 5-, and 6-mers, as well as bipartite motifs with 3 to 4 bp specificity part separated by 5 to 6 bp gaps). In order to select motifs of interest, an initial round of motif evaluation is performed on a subset of longer contigs (500 kbp minimum) with sufficient coverage (10×; Table 4; contigs from Bin 3, Bin 4, and Bin 9 were not covered sufficiently due to the use of a different DNA extraction kit than the SMRT study) with the rationale that results will have a higher statistical power. Uninformative methylation features are filtered out by discarding the ones with small absolute current difference values across the initial contig set (<1.5 in our study; chosen based on our mock metagenome analysis) as well as the ones computed from fewer than 20 motif occurrences. Next, we additionally filtered out uninformative methylation features from bipartite motifs by removing methylation feature vectors with fewer than two significant features across the initial contig set (significant if current difference >=1.5) to account for the longer vector and generally lower motif frequency. Finally, methylation features from bipartite motifs that overlap with any remaining 4 to 6-mer motifs are also discarded. The resulting list of informative methylation features is then evaluated in each contig of the metagenome assembly to construct a methylation profile matrix. This two-step approach effectively reduces the initial research space on the set of large contigs speeding up the analysis, and reduces noise by only considering methylation features selected from contigs with higher statistical power. The resulting methylation profile matrix (significant methylation features computed across all contigs) is then processed using t-SNE dimensionality reduction method to visualize contig clusters (FIG. 12). Missing methylation features and ones computed from fewer than 5 motifs occurrences are set to 0, small contigs are not considered for methylation binning (<10 kbp), and remaining ones are weighted according to their length. Weighting factors are defined as quotient of contig length divided by 50,000 and capped at 5% of number of remaining contigs to avoid extreme imbalance (only contigs with coverage >=10× for both native and WGA are weighted).

Motif detection from bins is performed the same way than for individual bacteria. With de novo detected motifs, methylation feature vectors used for binning are not filtered keeping the full-length methylation feature vectors. Missing methylation feature from individual contigs are handled as described previously and contigs are also weighted. Confirmation of de novo discovered motifs (potential 6 mA and 4mC motifs) from nanopore sequencing analysis were realized with per bin motif detection from SMRT sequencing data using the SMRT portal pipeline (RS_Modification_and_Motif_Analysis.1). Binning focused on associating MGEs to host genome was performed using another metagenome reference from the SMRT study where binned contigs were replaced by per-bin reassemblies.

### (j) Detection of Metagenome Contigs Misassemblies

The rationale is to examine the consistency of methylation signal for a motif across different occurrence of the motif along a metagenomic contig. For every single motif occurrence, we calculate a score by taking the average of absolute current differences from six consecutives positions with the most perturbation. Then, these individual scores are averaged using a sliding window across the contig to examine the continuity. Motif occurrences from both strands are used in this analysis. However, if a motif occurrence overlaps with another motif site being examined (<15 bp) then both are discarded.

What is claimed is:

1. A computer-implemented method of deconvoluting metagenomic assembled contigs from a microbiome sample, the method comprising:
a) extracting DNA from the microbiome sample;
b) subjecting the extracted DNA to a single-molecule sequencing reaction using single-molecule sequencing technology to generate a raw signal;
c) processing the raw signal;
d) comparing the processed raw signal and a known raw signal, wherein the known raw signal is generated from a biomolecule consisting of matched sequence;
e) computing DNA modification feature vectors from deviation between processed raw signal and the known raw signal for at least one sequence motif in at least two metagenomic assembled contigs;
f) selecting DNA modification features predicting a DNA modification within the sequence motifs in at least one of the metagenomic assembled contigs; and
g) binning metagenomic assembled contigs according to similarity of DNA modification profile matrix into clusters.

2. The method of claim 1, wherein the DNA modification comprises at least one DNA modification type selected from the group of methylation, hydroxymethylation, phosphorothioates, glucosylation and hexosylation.

3. The method of claim 1, wherein step (c) comprises the steps of:
a) mapping the raw signal to a known sequence of canonical monomers; and
b) reinforcing the raw signal.

4. The method of claim 3, wherein the method of reinforcing raw signal is accomplished by at least one method selected from the group of normalization, filtering, outlier removal, and aggregation.

5. The method of claim 1, wherein step (f) comprises determining a filtering criteria wherein the filtering criteria comprises at least one criterion selected from the group of feature value, feature frequency within metagenomic assembled contig, metagenomic assembled contig length, metagenomic assembled contig coverage, or sequence motif length.

6. The method of claim 1, wherein step (g) comprises creating a DNA modification profile matrix comprised of at least one DNA modification feature vector for at least one sequence motif for at least two contigs.

7. The method of claim 1, wherein the DNA modification feature vector computed in step (e) is at least of length two.

8. The method of claim 1, wherein step (b) comprises subjecting the extracted DNA to a single-molecule sequencing reaction using nanopore sequencing technology to generate a raw signal.

9. The method of claim 1, wherein the deconvolution of metagenomic contigs from the microbiome sample is used to match at least one mobile genetic element to at least one host genome.

10. The method of claim 9, wherein the mobile genetic element comprises a plasmid, a transposon, or a bacteriophage comprising at least one sequence motif of interest.

11. The method of claim 1, wherein the microbiome sample comprises at least two genomes of individual microorganisms.

12. The method of claim 1, wherein the microbiome sample comprises at least one source, the source selected from the group of a protozoa, an animal, a human, or a plant.

13. The method of claim 12, wherein the deconvolution of metagenomic contigs from the microbiome sample is used to diagnose, treat, classify, or a combination thereof at least one disease.

14. The method of claim 1, wherein the microbiome sample comprises at least one source, the source selected from the group of soil, air, water, sediment, oil, or combinations thereof.

15. The method of claim 14, wherein the deconvolution of metagenomic contigs from the microbiome sample is used to determine at least one contamination of location of microbiome sample collection.


SEQUENCE LISTING (89 sequences)

SEQ ID NO: 1 - DNA, 5 bp, Artificial Sequence
  Synthetic sequence; Methylation motif (5mCNGC)
  misc_feature (3)..(3): n is t or u
gcngc

SEQ ID NO: 2 - DNA, 6 bp, Artificial Sequence
  Synthetic sequence; Methylation motif (GGAT4mCC)
ggatcc

SEQ ID NO: 3 - DNA, 4 bp, Artificial Sequence
  Synthetic sequence; Methylation motif (GAT5mC)
gatc

SEQ ID NO: 4 - DNA, 4 bp, Artificial Sequence
  Synthetic sequence; Methylation motif (5mCCGG)
ccgg

SEQ ID NO: 5 - DNA, 13 bp, Artificial Sequence
  Synthetic sequence; Methylation motif (C6mACNNNNNNTAAA)
  misc_feature (4)..(9): n is a, c, g, t or u
cacnnnnnntaaa

SEQ ID NO: 6 - DNA, 5 bp, Artificial Sequence
  Synthetic sequence; Methylation motif (GGN5mCC)
  misc_feature (3)..(3): n is t or u
ggncc

SEQ ID NO: 7 - DNA, 6 bp, Artificial Sequence
  Synthetic sequence; Methylation motif (GTAT6mAC)
gtatac

SEQ ID NO: 8 - DNA, 13 bp, Artificial Sequence
  Synthetic sequence; Methylation motif (TTT6mANNNNNNGTG)
  misc_feature (5)..(10): n is a, c, g, t or u
tttannnnnngtg

SEQ ID NO: 9 - DNA, 6 bp, Artificial Sequence
  Synthetic sequence; Methylation motif (NGAC6mAT)
  misc_feature (1)..(1): n is c or g
ngacat

SEQ ID NO: 10 - DNA, 13 bp, Artificial Sequence
  Synthetic sequence; Methylation motif (A6mACNNNNNNGTGC)
  misc_feature (4)..(9): n is a, c, g, t or u
aacnnnnnngtgc

SEQ ID NO: 11 - DNA, 5 bp, Artificial Sequence
  Synthetic sequence; Methylation motif (C5mCNGG)
  misc_feature (3)..(3): n is t or u
ccngg

SEQ ID NO: 12 - DNA, 13 bp, Artificial Sequence
  Synthetic sequence; Methylation motif (GC6mACNNNNNNGT)
  misc_feature (5)..(10): n is a, c, g, t or u
gcacnnnnnngtt

SEQ ID NO: 13 - DNA, 6 bp, Artificial Sequence
  Synthetic sequence; Methylation motif (ATTA6mAT)
attaat

SEQ ID NO: 14 - DNA, 4 bp, Artificial Sequence
  Synthetic sequence; Methylation motif (C6mATG)
catg

SEQ ID NO: 15 - DNA, 13 bp, Artificial Sequence
  Synthetic sequence; Methylation motif (CNT6mANNNNNNNNC)
  misc_feature (2)..(2): n is a or g
  misc_feature (5)..(12): n is a, c, g, t or u
cntannnnnnnnc

SEQ ID NO: 16 - DNA, 4 bp, Artificial Sequence
  Synthetic sequence; Methylation motif (CN6mAG)
  misc_feature (2)..(2): n is c or g
cnag

SEQ ID NO: 17 - DNA, 6 bp, Artificial Sequence
  Synthetic sequence; Methylation motif (CTNN6mAG)
  misc_feature (3)..(4): n is a, c, g, t or u
ctnnag

SEQ ID NO: 18 - DNA, 12 bp, Artificial Sequence
  Synthetic sequence; Methylation motif (CN6mANNNNNNTTC)
  misc_feature (2)..(2): n is c, t or u
  misc_feature (4)..(9): n is a, c, g, t or u
cnannnnnnttc

SEQ ID NO: 19 - DNA, 4 bp, Artificial Sequence
  Synthetic sequence; Methylation motif (G5mCGC)
gcgc

SEQ ID NO: 20 - DNA, 4 bp, Artificial Sequence
  Synthetic sequence; Methylation motif (G6mAGG)
gagg

SEQ ID NO: 21 - DNA, 13 bp, Artificial Sequence
  Synthetic sequence; Methylation motif (G6mANNNNNNNTANG)
  misc_feature (3)..(9): n is a, c, g, t or u
  misc_feature (12)..(12): n is c, t or u
gannnnnnntang

SEQ ID NO: 22 - DNA, 12 bp, Artificial Sequence
  Synthetic sequence; Methylation motif (GA6mANNNANNTNG)
  misc_feature (4)..(9): n is a, c, g, t or u
  misc_feature (11)..(11): n is a or g
gaannnnnntng

SEQ ID NO: 23 - DNA, 6 bp, Artificial Sequence
  Synthetic sequence; Methylation motif (GA6mATTC)
gaattc

SEQ ID NO: 24 - DNA, 4 bp, Artificial Sequence
  Synthetic sequence; Methylation motif (GG5mCC)
ggcc

SEQ ID NO: 25 - DNA, 5 bp, Artificial Sequence
  Synthetic sequence; Methylation motif (GNNG6mA)
  misc_feature (2)..(3): n is a, c, g, t or u
gnnga

SEQ ID NO: 26 - DNA, 4 bp, Artificial Sequence
  Synthetic sequence; Methylation motif (GT6mAC)
gtac

SEQ ID NO: 27 - DNA, 6 bp, Artificial Sequence
  Synthetic sequence; Methylation motif (GTNN6mAC)
  misc_feature (3)..(4): n is a, c, g, t or u
gtnnac

SEQ ID NO: 28 - DNA, 5 bp, Artificial Sequence
  Synthetic sequence; Methylation motif (T4mCTTC)
tcttc

SEQ ID NO: 29 - DNA, 4 bp, Artificial Sequence
  Synthetic sequence; Methylation motif (TCG6mA)
tcga

SEQ ID NO: 30 - DNA, 6 bp, Artificial Sequence
  Synthetic sequence; Methylation motif (TCNNG6mA)
  misc_feature (3)..(4): n is a, c, g, t or u
tcnnga

SEQ ID NO: 31 - DNA, 4 bp, Artificial Sequence
  Synthetic sequence; Methylation motif (TGC6mA)
tgca

SEQ ID NO: 32 - DNA, 5 bp, Artificial Sequence
  Synthetic sequence; Methylation motif (4mCTNAG)
  misc_feature (3)..(3): n is a, c, g, t or u
ctnag

SEQ ID NO: 33 - DNA, 4 bp, Artificial Sequence
  Synthetic sequence; Methylation motif (AG4mCT)
agct

SEQ ID NO: 34 - DNA, 6 bp, Artificial Sequence
  Synthetic sequence; Methylation motif (CCA4mCGN)
  misc_feature (6)..(6): n is g, t or u
ccacgn

SEQ ID NO: 35 - DNA, 7 bp, Artificial Sequence
  Synthetic sequence; Methylation motif (GCNNG6mAT)
  misc_feature (3)..(4): n is c, t or u
gcnngat

SEQ ID NO: 36 - DNA, 6 bp, Artificial Sequence
  Synthetic sequence; Methylation motif (C5mCGCGG)
ccgcgg

SEQ ID NO: 37 - DNA, 6 bp, Artificial Sequence
  Synthetic sequence; Methylation motif (G5mCCGGC)
gccggc

SEQ ID NO: 38 - DNA, 11 bp, Artificial Sequence
  Synthetic sequence; Methylation motif (G6mAGNNNNNTAC)
  misc_feature (4)..(8): n is a, c, g, t or u
gagnnnnntac

SEQ ID NO: 39 - DNA, 14 bp, Artificial Sequence
  Synthetic sequence; Methylation motif (GC6mANNNNNNNNTGC)
  misc_feature (4)..(11): n is a, c, g, t or u
gcannnnnnnntgc

SEQ ID NO: 40 - DNA, 6 bp, Artificial Sequence
  Synthetic sequence; Methylation motif (GGNN5mCC)
  misc_feature (3)..(4): n is a, c, g, t or u
ggnncc

SEQ ID NO: 41 - DNA, 5 bp, Artificial Sequence
  Synthetic sequence; Methylation motif (GGTG6mA)
ggtga

SEQ ID NO: 42 - DNA, 11 bp, Artificial Sequence
  Synthetic sequence; Methylation motif (GT6mANNNNNCTC)
  misc_feature (4)..(8): n is a, c, g, t or u
gtannnnnctc

SEQ ID NO: 43 - DNA, 6 bp, Artificial Sequence
  Synthetic sequence; Methylation motif (NG5mCGCN)
  misc_feature (1)..(1): n is a or a
  misc_feature (6)..(6): n is c, t or u
ngcgcn

SEQ ID NO: 44 - DNA, 6 bp, Artificial Sequence
  Synthetic sequence; Methylation motif (G5mCCGGC)
gccggc

SEQ ID NO: 45 - DNA, 6 bp, Artificial Sequence
  Synthetic sequence; Methylation motif
  misc_feature (3)..(3): n is t or u
ggntaa

SEQ ID NO: 46 - DNA, 6 bp, Artificial Sequence
  Synthetic sequence; Methylation motif
  misc_feature (3)..(3): n is t or u
  misc_feature (5)..(5): n is a, c, g, t or u
ggncna

SEQ ID NO: 47 - DNA, 11 bp, Artificial Sequence
  Synthetic sequence; Methylation motif
  misc_feature (4)..(8): n is a, c, g, t or u
gtannnnnccc

SEQ ID NO: 48 - DNA, 11 bp, Artificial Sequence
  Synthetic sequence; Methylation motif (GT6mANNNNNCTC)
  misc_feature (4)..(8): n is a, c, g, t or u
gtannnnnctc

SEQ ID NO: 49 - DNA, 5 bp, Artificial Sequence
  Synthetic sequence; Methylation motif (T5mCACC)
tcacc

SEQ ID NO: 50 - DNA, 6 bp, Artificial Sequence
  Synthetic sequence; Methylation motif
  misc_feature (1)..(1): n is t or u
  misc_feature (6)..(6): n is a or g
nggccn

SEQ ID NO: 51 - DNA, 6 bp, Artificial Sequence
  Synthetic sequence; Methylation motif
  misc_feature (1)..(1): n is t or u
  misc_feature (6)..(6): n is t or u
nggccn

SEQ ID NO: 52 - DNA, 6 bp, Artificial Sequence
  Synthetic sequence; Methylation motif (CTGG6mAG)
ctggag

SEQ ID NO: 53 - DNA, 7 bp, Artificial Sequence
  Synthetic sequence; Methylation motif (CCTCT6mAG)
cctctag

SEQ ID NO: 54 - DNA, 7 bp, Artificial Sequence
  Synthetic sequence; Methylation motif (NTA6mATTC)
  misc_feature (1)..(1): n is c or g
ntaattc

SEQ ID NO: 55 - DNA, 5 bp, Artificial Sequence
  Synthetic sequence; Methylation motif (CC6mACC)
ccacc

SEQ ID NO: 56 - DNA, 6 bp, Artificial Sequence
  Synthetic sequence; Methylation motif (ACCG5mCG)
accgag

SEQ ID NO: 57 - DNA, 5 bp, Artificial Sequence
  Synthetic sequence; Methylation motif (A5mCGGG)
acggg

SEQ ID NO: 58 - DNA, 5 bp, Artificial Sequence
  Synthetic sequence; Methylation motif (C5mCCGT)
cccgt

SEQ ID NO: 59 - DNA, 6 bp, Artificial Sequence
  Synthetic sequence; Methylation motif (NC5mCGGN)
  misc_feature (1)..(1): n is g, t or u
  misc_feature (6)..(6): n is a or c
nccggn

SEQ ID NO: 60 - DNA, 6 bp, Artificial Sequence
  Synthetic sequence; Methylation motif (N5mCCGGN)
  misc_feature (1)..(1): n is t or u
  misc_feature (6)..(6): n is t or u
nccggn

SEQ ID NO: 61 - DNA, 5 bp, Artificial Sequence
  Synthetic sequence; Methylation motif (AG5mCTC)
agctc

SEQ ID NO: 62 - DNA, 5 bp, Artificial Sequence
  Synthetic sequence; Methylation motif (CGN5mCG)
  misc_feature (3)..(3): n is t or u
cgncg

SEQ ID NO: 63 - DNA, 6 bp, Artificial Sequence
  Synthetic sequence; Methylation motif (CTG6mAAG)
ctgcag

SEQ ID NO: 64 - DNA, 5 bp, Artificial Sequence
  Synthetic sequence; Methylation motif (GAG5mCT)
gagct

SEQ ID NO: 65 - DNA, 6 bp, Artificial Sequence
  Synthetic sequence; Methylation motif (N5mCCGGN)
  misc_feature (1)..(1): n is a or g
  misc_feature (6)..(6): n is c, t or u
nccggn

SEQ ID NO: 66 - DNA, 13 bp, Artificial Sequence
  Synthetic sequence; Methylation motif (AGC6mANNNNNNNT)
  misc_feature (5)..(11): n is a, c, g, t or u
agcannnnnnntc

SEQ ID NO: 67 - DNA, 13 bp, Artificial Sequence
  Synthetic sequence; Methylation motif (G6mANNNNNNNTGCT)
  misc_feature (3)..(9): n is a, c, g, t or u
gannnnnnntgct

SEQ ID NO: 68 - DNA, 6 bp, Artificial Sequence
  Synthetic sequence; Methylation motif (AA6mAAGC)
aacagc

SEQ ID NO: 69 - DNA, 6 bp, Artificial Sequence
  Synthetic sequence; Methylation motif (ATGC6mAT)
atgcat

SEQ ID NO: 70 - DNA, 11 bp, Artificial Sequence
  Synthetic sequence; Methylation motif (6mANCNNNNNTAG)
  misc_feature (2)..(2): n is c, t or u
  misc_feature (4)..(8): n is a, c, g, t or u
ancnnnnntag

SEQ ID NO: 71 - DNA, 5 bp, Artificial Sequence
  Synthetic sequence; Methylation motif (C5mCNGG)
  misc_feature (3)..(3): n is a, c, g, t or u
ccngg

SEQ ID NO: 72 - DNA, 5 bp, Artificial Sequence
  Synthetic sequence; Methylation motif (CGN5mCG)
  misc_feature (3)..(3): n is a, t or u
cgncg

SEQ ID NO: 73 - DNA, 11 bp, Artificial Sequence
  Synthetic sequence; Methylation motif (CT6mANNNNNGNT)
  misc_feature (4)..(8): n is a, c, g, t or u
  misc_feature (10)..(10): n is a or g
ctannnnngnt

SEQ ID NO: 74 - DNA, 6 bp, Artificial Sequence
  Synthetic sequence; Methylation motif (GAG5mCCC)
gagccc

SEQ ID NO: 75 - DNA, 6 bp, Artificial Sequence
  Synthetic sequence; Methylation motif (GAG5mCTC)
gagctc

SEQ ID NO: 76 - DNA, 6 bp, Artificial Sequence
  Synthetic sequence; Methylation motif (GGG5mCTC)
gggctc

SEQ ID NO: 77 - DNA, 5 bp, Artificial Sequence
  Synthetic sequence; Methylation motif (GGN5mCC)
  misc_feature (3)..(3): n is a, c, g, t or u
ggncc

SEQ ID NO: 78 - DNA, 14 bp, Artificial Sequence
  Synthetic sequence; Methylation motif (AC6mANNNNNNNNTGG)
  misc_feature (4)..(11): n is a, c, g, t or u
acannnnnnnntgg

SEQ ID NO: 79 - DNA, 14 bp, Artificial Sequence
  Synthetic sequence; Methylation motif (CC6mANNNNNNNNTGT)
  misc_feature (4)..(11): n is a, c, g, t or u
ccannnnnnnntgt

SEQ ID NO: 80 - DNA, 5 bp, Artificial Sequence
  Synthetic sequence; Methylation motif (C5mCAGA)
ccaga

SEQ ID NO: 81 - DNA, 6 bp, Artificial Sequence
  Synthetic sequence; Methylation motif (GG6mAAGC)
ggaagc

SEQ ID NO: 82 - DNA, 5 bp, Artificial Sequence
  Synthetic sequence; Methylation motif (GGN5mCC)
  misc_feature (3)..(3): n is a, c, g, t or u
ggncc

SEQ ID NO: 83 - DNA, 6 bp, Artificial Sequence
  Synthetic sequence; Methylation motif (GTG6mATG)
gtgatg

SEQ ID NO: 84 - DNA, 5 bp, Artificial Sequence
  Synthetic sequence; Methylation motif (T5mCTGG)
tctgg

SEQ ID NO: 85 - DNA, 5 bp, Artificial Sequence
  Synthetic sequence; Methylation motif (AG6mATG)
agatg

SEQ ID NO: 86 - DNA, 5 bp, Artificial Sequence
  Synthetic sequence; Methylation motif (C5mCCGC)
cccgc

SEQ ID NO: 87 - DNA, 5 bp, Artificial Sequence
  Synthetic sequence; Methylation motif (C5mCNGA)
  misc_feature (3)..(3): n is a, t or u
ccnga

SEQ ID NO: 88 - DNA, 5 bp, Artificial Sequence
  Synthetic sequence; Methylation motif (G5mCGGG)
gcggg

SEQ ID NO: 89 - DNA, 5 bp, Artificial Sequence
  Synthetic sequence; Methylation motif (T5mCNGG)
  misc_feature (3)..(3): n is a, t or u
tcngg